(19) United States
(12) Patent Application Publication
(10) Pub. No.: US 2016/0331446 A1
(43) Pub. Date: Nov. 17, 2016

(54) METHODS, DEVICES AND SYSTEMS FOR CAROTID BODY ABLATION VIA A TRANSRADIAL OR TRANSBRACHIAL APPROACH

(71) Applicant: CIBIEM, INC., Los Altos, CA (US)

(72) Inventors: Kenneth M. MARTIN, Woodside, CA (US); Mark GELFAND, New York, NY (US); Michael Brick MARKHAM, Redwood City, CA (US)

(21) Appl. No.: 15/105,599

(22) PCT Filed: Jan. 5, 2015
(86) PCT No.: PCT/US2015/010156

§ 371 Date: Jun. 17, 2016

Related U.S. Application Data
(60) Provisional application No. 61/924,067, filed on Jan. 6, 2014.

(51) Int. Cl. A61B 18/14

(52) U.S. Cl. CPC A61B 18/1492; A61B 2018/00202

(57) ABSTRACT

Described herein are methods and endovascular catheters, configured for delivery to a carotid artery from a subclavian artery approach, for assessing, and treating patients having sympathetically mediated disease, involving augmented peripheral chemoreflex and heightened sympathetic tone by reducing chemosensor input to the nervous system via carotid body ablation.

### CROSS REFERENCE TO RELATED APPLICATIONS

This application claims priority to U.S. Prov. App. No. 61/924,067, filed Jan. 6, 2014, which is incorporated herein by reference.

The following applications are incorporated herein by reference: U.S. Prov. App. No. 61/667,991, filed Jul. 4, 2012; U.S. Prov. App. No. 61/667,996, filed Jul. 4, 2012; U.S. Prov. App. No. 61/667,998, filed Jul. 4, 2012; U.S. Prov. App. No. 61/682,034, filed Aug. 10, 2012; U.S. Prov. App. No. 61/768,101, filed Feb. 22, 2013; U.S. Prov. App. No. 61/791,769, filed Mar. 15, 2013; U.S. Prov. App. No. 61/791,420, filed Mar. 15, 2013; U.S. Prov. App. No. 61/792,214, filed Mar. 15, 2013; U.S. Prov. App. No. 61/792,741, filed Mar. 15, 2013; U.S. Prov. App. No. 61/793,267, filed Mar. 15, 2013; U.S. Prov. App. No. 61/794,667, filed Mar. 15, 2013; U.S. Prov. App. No. 61/810,639, filed Apr. 10, 2013; and U.S. Prov. App. No. 61/836,100, filed Jun. 17, 2013; and U.S. application Ser. No. 13/936,121, filed Jul. 5, 2013.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure is directed generally to devices, systems and methods for treating patients having sympathetically mediated disease associated at least in part with augmented peripheral chemoreflex, heightened sympathetic activation, or autonomic imbalance by ablating at least one peripheral chemoreceptor (e.g., a carotid body) or an associated nerve.

### BACKGROUND

It is known that an imbalance of the autonomic nervous system is associated with several disease states. Restoration of autonomic balance has been a target of several medical treatments including modalities such as pharmacological, device-based, and electrical stimulation. For example, beta blockers are a class of drugs used to reduce sympathetic activity to treat cardiac arrhythmias and hypertension; Gelfand and Levin (U.S. Pat. No. 7,162,303) describe a device-based treatment used to decrease renal sympathetic activity to treat heart failure, hypertension, and renal failure; Yun and Yuarn-Bor (U.S. Pat. No. 7,149,574; U.S. Pat. No. 7,363,076; U.S. Pat. No. 7,738,952) describe a method of restoring autonomic balance by increasing parasympathetic activity to treat disease associated with parasympathetic attrition; Kieval, Burns and Serdar (U.S. Pat. No. 8,060,206) describe an electrical pulse generator that stimulates a baroreceptor, increasing parasympathetic activity, in response to high blood pressure; Hlavka and Elliott (US 2010/0070004) describe an implantable electrical stimulator in communication with an afferent neural pathway of a carotid body chemoreceptor to control dyspnea via electrical neuromodulation. US 2012/0172680 describes carotid body ablation for treating sympathetically mediated diseases.

Ablating a carotid body in a human patient is risky and difficult. A carotid body is typically about the size of a grain of rice, located near other glands, nerves, muscles and other organs, and moves with movement of the jaw and neck, respiration and blood pulsation. Conventional open surgical techniques to access the carotid body directly through the neck that are referred to as open surgery are challenging due to the nerves, muscles, arteries, veins and other organs near the carotid body. In the modern medicine open surgery is only used to access a carotid body for removal of carotid body tumors that are immediately life threatening.

### SUMMARY

Methods, devices and systems have been conceived for endovascular carotid body ablation with a catheter introduced through a radial or brachial artery to a common carotid artery.

There is a desire for minimally invasive surgical techniques and instruments configured to ablate at least a portion of the carotid body. Endovascular catheter assemblies are known for performing minimally invasive procedures and surgeries, including endovascular ablation of nerves, on the heart, kidney, pulmonary artery, renal artery and other body organs typically located below the neck. These catheter assemblies tend to be too short, too large, lack necessary features needed for retention and targeting of energy delivery and otherwise not suited to reaching the neck and, particularly, the narrow blood vessels in the neck. Endovascular catheter assemblies are also known for treating arteries in the neck such as to treat aneurysms in the wall of a blood vessel.

It is not conventional to use traditional minimally invasive surgical ablation instruments and techniques to treat organs in the neck, particularly at and near the bifurcation of carotid artery where the carotid body is located. One difficulty with applying endovascular catheter ablation techniques to an organ in the neck, other than an artery or vein in the torso or abdomen, is the long and tortuous path through the vascular system that a catheter is generally advanced to reach the neck. Another difficulty can be properly positioning the distal end of the catheter in an artery to act on the target organ that is external to the artery. Another difficulty is avoiding damage to carotid endothelium that can lead to formation of thrombus, avoiding excessive heating and scarring of blood vessel walls that can lead to stenosis, or disturbing atherosclerotic plaque that can cause embolization of brain arteries and stroke. The organ may move with respect to the artery, the narrow arteries in the neck and the complex geometries of these arteries present challenges to a minimally invasive technique to reach the carotid body. Ablation procedures may take tens of seconds and even minutes and in the highly mobile are of the neck catheter can be displaced during energy application.

While catheter probes with stimulation electrodes have been proposed for electrically stimulating the carotid body, these approaches do not describe ablating or otherwise permanently changing the carotid body. Nor do they describe devices and systems that are used to accomplish the same. Ablating, modulating or otherwise permanently changing the carotid body or its function requires the application of energy, chemicals or other forces sufficient to damage the carotid body or its associated nerves and potentially tissue and blood vessel walls near the carotid body. Damaging the carotid body, nerves and nearby tissue is not necessary or desired if the object of a treatment is to electrically stimulate the carotid body. Applying a relatively low level of energy to electrically stimulate the carotid body will unlikely damage a blood vessel or surrounding tissue, even if the energy is applied to a broader area than the carotid body. The level of energy and force or the chemicals needed to ablate the carotid body is substantially higher than the levels needed for stimulation. Applying energy, chemicals and forces (e.g., thermal energy) sufficient to damage the carotid body raises concerns that the damage could extend to nearby non-target nerves and other organs, rupture the wall of the blood vessel, disturb and dislodge plaque or create blood clots that could flow to the brain.

In view of the need to damage the carotid body, there are strict requirements for positioning and retaining the tip of an ablating catheter in a carotid artery for the duration of the procedure, and for narrowly targeting the delivery of the energy, chemicals or force to the carotid body. Recognizing and identifying the requirements for positioning an ablating tip, or energy application element, of a catheter was a first step for an endovascular catheter assembly for ablating the carotid body. A second step included the invention of endovascular catheter assemblies that satisfied the requirements. Then parameters for energy application were developed that preserve the blood vessel and surrounding non-target tissues but substantially ablate the carotid body or an associated nerve.

A function of a carotid body may be stimulated (e.g., excited with electric signal or chemical) and at least one physiological parameter is recorded prior to and during the stimulation, then the carotid body is ablated, and the stimulation is repeated, whereby the change in recorded physiological parameter(s) prior to and after ablation is an indication of the effectiveness of the ablation.

A function of a carotid body may be temporarily blocked and at least one physiological parameter(s) is recorded prior to and during the blockade, then the carotid body is ablated, and the blockade is repeated, whereby the change in recorded physiological parameter(s) prior to and after ablation is an indication of the effectiveness of the ablation.

A device configured to prevent embolic debris from entering the brain may be deployed in an internal carotid artery associated with a carotid body, then an ablation element is placed within and against the wall of an external carotid artery or an internal carotid artery associated with the carotid body, the ablation element is activated resulting in carotid body ablation, the ablation element is then withdrawn, then the embolic prevention device is withdrawn, whereby the embolic prevention device in the internal carotid artery prevents debris resulting from the use of the ablation element form entering the brain.

A system has been conceived comprising a vascular catheter configured with an ablation element in the vicinity of the distal end, and a connection between the ablation element and a source of ablation energy at the proximal end, whereby the distal end of the catheter is constructed to be inserted into a peripheral artery of a patient and then maneuvered into an internal or external carotid artery using standard fluoroscopic guidance techniques and positioned in a predetermined position at a carotid bifurcation.

A system has been conceived comprising a vascular catheter configured with an ablation element in vicinity of a distal end configured for carotid body ablation and further configured for at least one of the following: neural stimulation, neural blockade, carotid body stimulation and carotid body blockade; and a connection between the ablation element and a source of ablation energy, stimulation energy and/or blockade energy.

A system has been conceived comprising a vascular catheter configured with an ablation element and at least one electrode configured for at least one of the following: neural stimulation, neural blockade, carotid body stimulation and carotid body blockade; and a connection between the ablation element to a source of ablation energy, and a connection between the ablation element and/or electrode(s) to a source of stimulation energy and/or blockade energy.

A system for endovascular transmural ablation of a carotid body has been conceived comprising a carotid artery catheter with an ablation element mounted on a distal region of the catheter, a means for pressing the ablation element against a wall of a carotid artery at a specific location, a means for connecting the ablation element to a source of ablation energy mounted at a proximal region of the catheter, and a console comprising a source of ablation energy, a means for controlling the ablation energy, a user interface configured to provide the user with a selection of ablation parameters, indications of the status of the console and the status of the ablation activity, a means to activate and deactivate an ablation, and an umbilical to provide a means for connecting the catheter to the console.

A method has been conceived to reduce or inhibit chemoreflex generated by a carotid body in a human patient, to reduce afferent nerve sympathetic activity of carotid body nerves to treat a sympathetically mediated disease, the method comprising: positioning a catheter in a vascular system of the patient such that a distal section of the catheter is in a lumen proximate to a carotid body of the patient; pressing an ablation element against a wall of the lumen adjacent to the carotid body, supplying energy to the ablation element wherein the energy is supplied by an energy supply apparatus outside of the patient; applying the energy from the energy supply to the ablation element to ablate tissue proximate to or included in the carotid body; and removing the ablation device from the patient; wherein a carotid body chemoreflex function is inhibited or sympathetic afferent nerve activity of carotid body nerves is reduced due to the ablation.

A method has been conceived to treat a patient having a sympathetically mediated disease by reducing or inhibiting chemoreflex function generated by a carotid body including steps of inserting a catheter into the patient's vasculature, positioning a portion of the catheter proximate a carotid body (e.g., in a carotid artery), positioning an ablation element toward a target ablation site (e.g., carotid body, intercarotid septum, carotid plexus, carotid body nerves, carotid sinus nerve), holding position of the catheter, applying ablative energy to the target ablation site via the ablation element, and removing the catheter from the patient's vasculature, wherein the ablative energy is sufficient to cool or heat tissue sufficiently to substantially reduce chemoreflex or afferent nerve signals from the carotid body while avoiding ablation of nearby important non-target nerve structures.

The methods and systems disclosed herein may be applied to satisfy clinical needs related to treating cardiac, metabolic, and pulmonary diseases associated, at least in part, with augmented chemoreflex (e.g., high chemosensor sensitivity or high chemosensor activity) and related sympathetic activation. The treatments disclosed herein may be used to restore autonomic balance by reducing sympathetic activity, as opposed to increasing parasympathetic activity. It is understood that parasympathetic activity can increase as a result of the reduction of sympathetic activity (e.g., sympathetic withdrawal) and normalization of autonomic balance. Furthermore, the treatments may be used to reduce sympathetic activity by modulating a peripheral chemoreflex. Furthermore, the treatments may be used to reduce afferent neural stimulus, conducted via afferent carotid body nerves, from a carotid body to the central nervous system. Enhanced peripheral and central chemoreflex is implicated in several pathologies including hypertension, cardiac tachyarrhythmias, sleep apnea, dyspnea, chronic obstructive pulmonary disease (COPD), diabetes and insulin resistance, and CHF. Mechanisms by which these diseases progress may be different, but they may commonly include contribution from increased afferent neural signals from a carotid body. Central sympathetic nervous system activation is common to all these progressive and debilitating diseases. Peripheral chemoreflex may be modulated, for example, by modulating carotid body activity. The carotid body is the sensing element of the afferent limb of the peripheral chemoreflex. Carotid body activity may be modulated, for example, by substantially ablating a carotid body or afferent nerves emerging from the carotid body. Such nerves can be found in a carotid body itself, in a carotid plexus, in an intercarotid septum, in periarterial space of a carotid bifurcation and internal and external carotid arteries. Therefore, a therapeutic method has been conceived that comprises a goal of restoring or partially restoring autonomic balance by reducing or removing carotid body input into the central nervous system.

### BRIEF DESCRIPTION OF THE DRAWINGS

FIG. 1 is a lateral view illustrating a patient's left intercarotid septum.

FIG. 2 is a transverse cross sectional view illustrating a patient's intercarotid septum.

FIG. 3A is a schematic view of a steerable sheath.

FIG. 3B is a schematic view of a steerable sheath in a deflected state.

FIGS. 4A, 4B, and 4C are schematic illustrations of human aortic arch anatomy.

FIGS. 5A, 5B, 5C, and 5D are schematic illustrations of a method of delivering a guidewire over an acute vascular angle.

FIGS. 6A and 6B are schematic illustrations of a folded guidewire.

FIG. 7 is a schematic illustration of a guidewire delivery sheath having a hinged distal end.

FIGS. 8A and 8B are schematic illustrations of a sheath configured to increase a diameter of curvature of an acute vascular transition.

FIGS. 9A and 9B are schematic illustrations of a carotid body ablation catheter delivered over two guidewires.

FIG. 10 is a schematic illustration of a carotid body ablation catheter with a rotatable distal structure.

FIG. 11 illustrates an exemplary carotid body ablation catheter.

FIGS. 12A to 12G are illustrations of electrodes.

FIGS. 13A and 13B are schematic views showing suitable placement of ablation elements on an intercarotid septum.

FIGS. 14A, 14B, and 14C are illustrations of an Endovascular Transmural Ablation Precision-Grip catheter configured for controllable deflection with a slide-on arm configuration in use.

FIG. 15 is a schematic illustration of an Endovascular Transmural Ablation Precision-Grip catheter configured for monopolar ablation and intercarotid septum monitoring.

### DETAILED DESCRIPTION

In the following detailed description, reference is made to the accompanying drawings, which form a part hereof, and in which is shown by way of illustration exemplary embodiments in which the disclosure may be practiced. These embodiments are described in sufficient detail to enable those skilled in the art to practice the inventions, and it is to be understood that the embodiments may be combined, or that other embodiments may be utilized and that structural, logical and electrical changes may be made without departing from the spirit and scope of the present disclosure.

References to “an”, “one”, or “various” embodiments in this disclosure are not necessarily to the same embodiment, and such references contemplate more than one embodiment. The following detailed description provides exemplary embodiments.

Systems, devices, and methods have been conceived for carotid body ablation (that is, full or partial ablation of one or both carotid bodies, carotid body nerves, intercarotid septums, or peripheral chemoreceptors) to treat patients having a sympathetically mediated disease (e.g., cardiac, renal, metabolic, or pulmonary disease such as hypertension, congestive heart failure, atrial fibrillation, ventricular tachycardia, dyspnea, sleep apnea, sleep disordered breathing, diabetes, insulin resistance, atrial fibrillation, chronic kidney disease, polycystic ovarian syndrome, post MI mortality) at least partially resulting from augmented peripheral chemoreflex (e.g., peripheral chemoreceptor hypersensitivity, peripheral chemosensor hyperactivity), heightened sympathetic activation, or an unbalanced autonomic tone.

A reduction of peripheral chemoreflex or reduction of afferent nerve signaling from a carotid body (CB) resulting in a reduction of central sympathetic tone is a main therapy pathway of the methods described herein. Higher than normal chronic or intermittent activity of afferent carotid body nerves is considered enhanced chemoreflex. Other therapeutic benefits such as increase of parasympathetic tone, vagal tone and specifically baroreflex and baroreceptor activity, as well as reduction of dyspnea, hyperventilation, hypercapnea, respiratory alkalosis and breathing rate may be expected in some patients. Secondary to reduction of breathing rate additional increase of parasympathetic tone can be expected in some patients. Reduced breathing rate can lead to increased tidal lung volume, reduced dead space and increased efficiency of gas exchange. Reduced dyspnea and reduced dead space can independently lead to improved ability to exercise. Shortness of breath (dyspnea) and exercise limitations are common debilitating symptoms in CHF and COPD. Augmented peripheral chemoreflex (e.g., carotid body activation) leads to increases in sympathetic nervous system activity, which is in turn primarily responsible for the progression of chronic disease as well as debilitating symptoms and adverse events seen in our intended patient populations. Carotid bodies contain cells that are sensitive to partial pressure of oxygen and carbon dioxide in blood plasma. Carotid bodies also may respond to blood flow, pH acidity, glucose level in blood and possibly other variables. Thus carotid body ablation may be a treatment for patients, for example having hypertension, heart disease or diabetes, even if chemosensitive cells are not activated.

The disclosure herein includes methods of a subclavian approach for endovascular carotid body ablation, which in some embodiments include inserting a catheter in the patient's vascular system, positioning a distal region of the catheter in a vessel proximate a carotid body (e.g., in a common carotid artery, internal carotid artery, external carotid artery, at a carotid bifurcation, proximate an intercarotid septum), coupling the distal region of the catheter to a carotid bifurcation, positioning an ablation element proximate to a target site (e.g., a carotid body, afferent nerves associated with a carotid body, a peripheral chemosensor, an intercarotid septum), and delivering an ablation agent from the ablation element to ablate the target site. Exemplary methods and devices configured to perform these methods are described herein.

### Targets:

To inhibit or suppress a peripheral chemoreflex, anatomical targets for ablation (also referred to as target tissue, targeted tissue, target ablation sites, or target sites) may include at least a portion of at least one carotid body, an aortic body, nerves associated with a peripheral chemoreceptor (e.g., carotid body nerves, carotid sinus nerve, carotid plexus), small blood vessels feeding a peripheral chemoreceptor, carotid body parenchyma, chemosensitive cells (e.g., glomus cells), tissue in a location where a carotid body is suspected to reside (e.g., a location based on pre-operative imaging or anatomical likelihood), an intercarotid septum, a portion of an intercarotid septum or a combination thereof. As used herein, ablation of a carotid body or carotid body ablation may refer to ablation of any of these target ablation sites.

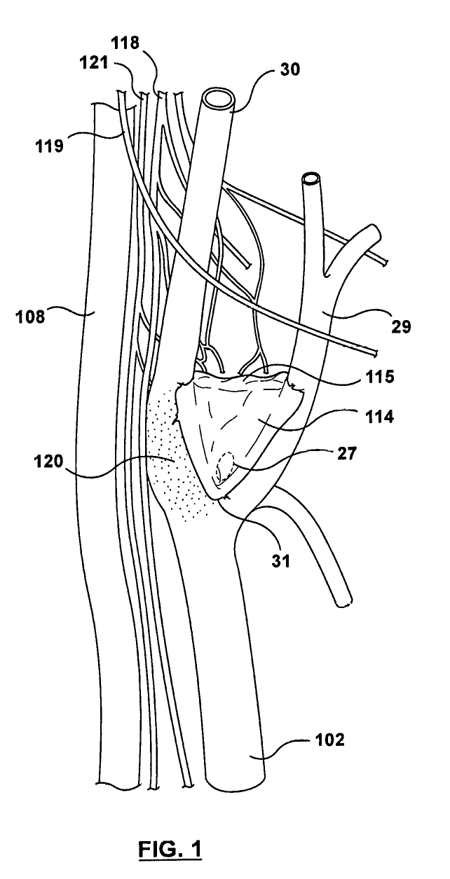

As shown in FIG. 1, a carotid body (“CB”) 27, housing peripheral chemoreceptors, modulates sympathetic tone through direct signaling to the central nervous system. Carotid bodies represent a paired organ system located near a bifurcation 31 of a common carotid artery 102 bilaterally, that is, on both sides of the neck. The common carotid artery 102 bifurcates to an internal carotid artery 30 and an external carotid artery 29. Typically, in humans each carotid body is approximately the size of a 2.5-5 mm ovoid grain of rice and is innervated both by the carotid sinus nerve (CSN, a branch of the glossopharyngeal nerve), and the ganglioglomerular (sympathetic) nerve of the nearby superior cervical ganglion. Infrequently other shapes are encountered. The CB is the most perfused organ per gram weight in the body and receives blood via an arterial branch or branches typically arising from internal or external carotid artery.

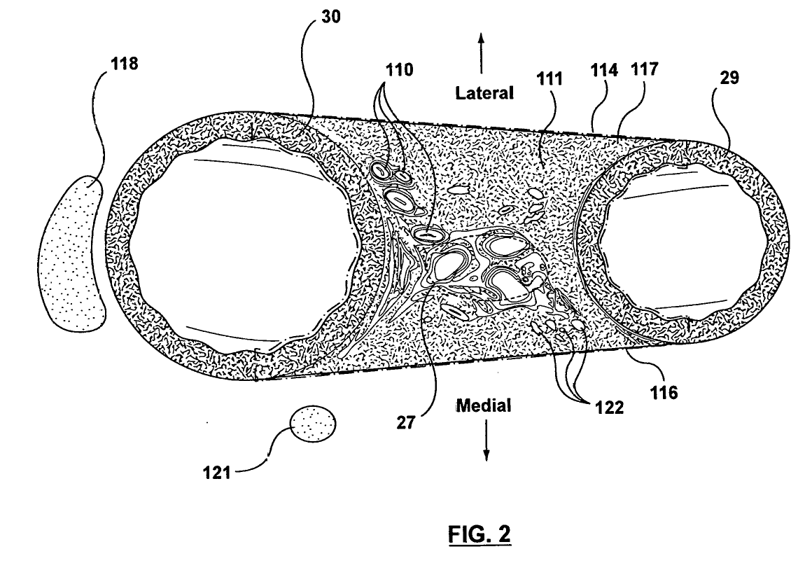

Inventors have conducted extensive human cadaver anatomy studies to understand variability in geometry and relative position of carotid arteries, carotid bodies, carotid nerves, and important non-target nerves. This information is an important part of the inventive step to determine aspects of a procedure and device that could effectively ablate a targeted tissue (e.g., carotid body, carotid body nerves, substantial portion of a carotid body) while safely avoiding iatrogenic injury of important non-target nerves. Inventors have discovered that a volume of tissue, which is referred to herein as an intercarotid septum, carotid septum, or septum, may be a suitable target for ablation in a carotid body ablation (“CBA”) procedure. Endovascular catheter assemblies, such as those described herein, were designed to be configured to ablate at least a significant portion of, and containing an ablation within or substantially within, an intercarotid septum. An exemplary intercarotid septum 114, shown in FIGS. 1 and 2, is herein defined as a wedge or triangular segment of tissue with the following boundaries: a saddle of a carotid bifurcation 31 defines a caudal aspect (an apex) of a carotid septum 114; facing walls of internal 30 and external 29 carotid arteries define two sides of a carotid septum; a cranial boundary 115 of a carotid septum extends between these arteries and may be defined as cranial to a carotid body but caudal to any important non-target nerve structures (e.g., hypoglossal nerve) that might be in the region, for example a cranial boundary may be about 7 mm to 15 mm (e.g., about 10 mm) from the saddle of the carotid bifurcation; medial 116 and lateral 117 walls of the carotid septum 114 are generally defined by planes approximately tangent to the internal and external carotid arteries; one of the planes is tangent to the lateral wall of the internal and external carotid arteries and the other plane is tangent to the medial walls of these arteries. An intercarotid septum is between the medial and lateral walls. The medial plane of an intercarotid septum may alternatively be defined as a carotid sheath on a medial side of a septum or within about 2 mm outside of the medial side of the carotid sheath. An intercarotid septum 114 may include a carotid body 27 and is typically absent of important non-target nerve structures such as a vagus nerve 118, important non-target sympathetic nerves 121, or a hypoglossal nerve 119 (see FIG. 1). Creating an ablation that is maintained or substantially maintained within an intercarotid septum may therefore effectively modulate (e.g., ablate) a carotid body while safely avoiding collateral damage of important non-target nerve structures. Probability of effectiveness may be increased as the percentage of the septum encompassed by an ablation, at the level of a carotid body or cranial to the carotid body, increases. An intercarotid septum may include some baroreceptors 120 or baroreceptor nerves. An intercarotid septum may also include small blood vessels 110, nerves 122 associated with the carotid body, and fat 111.

As used herein, a “wall” of an external or internal carotid artery, or any other vessel, is not limited to the endothelial layer, but includes any other tissue or non-tissue associated with the vessel. For example, a wall includes plaque or any other material deposited thereon. As used herein, a “wall” of a blood vessel is anything that at least partially defines the lumen through which blood flows. For example, when an electrode is in apposition with a wall of a blood vessel, it may be in contact with an endothelial layer, plaque, etc.

Carotid body nerves are anatomically defined herein as carotid plexus nerves 122 (see FIG. 2) and carotid sinus nerves. Carotid body nerves are functionally defined herein as nerves that conduct information from a carotid body to a central nervous system.

An ablation may be focused exclusively on targeted tissue, or be focused on the targeted tissue while safely ablating tissue proximate to the targeted tissue (e.g., to ensure the targeted tissue is ablated or as an approach to gain access to the targeted tissue). An ablation may be as big as a peripheral chemoreceptor (e.g., carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the glomus cells. An intercarotid plexus or carotid sinus nerve may be a target of ablation with an understanding that some baroreceptor nerves will be ablated together with carotid body nerves. Baroreceptors are distributed in the human arteries and have high degree of redundancy.

Tissue may be ablated to inhibit or suppress a chemoreflex of only one of a patient's two carotid bodies. Alternatively, a carotid body ablation procedure may involve ablating tissue to inhibit or suppress a chemoreflex of both of a patient's carotid bodies. For example a therapeutic method may include ablation of one carotid body, measurement of resulting chemosensitivity, sympathetic activity, respiration or other parameter related to carotid body hyperactivity and ablation of the second carotid body if needed to further reduce chemosensitivity following unilateral ablation. The decision to ablate one or both carotid bodies may be based on pre-procedure testing or on patient's anatomy.

An embodiment of a therapy may substantially reduce chemoreflex without excessively reducing the baroreflex of the patient. The proposed ablation procedure may be targeted to substantially spare the carotid sinus, baroreceptors distributed in the walls of carotid arteries (e.g., internal carotid artery), and at least some of the carotid sinus baroreceptor nerves that conduct signals from said baroreceptors. For example, the baroreflex may be substantially spared by targeting a limited volume of ablated tissue possibly enclosing the carotid body, tissues containing a substantial number of carotid body nerves, tissues located in periadventitial space of a medial segment of a carotid bifurcation, or tissue located at the attachment of a carotid body to an artery. Said targeted ablation is enabled by visualization of the area or carotid body itself, for example by CT, CT angiography, MRI, ultrasound sonography, IVUS, OCT, intracardiac echocardiography (ICE), trans-esophageal echocardiography (TEE), fluoroscopy, blood flow visualization, or injection of contrast, and positioning of an instrument in the carotid body or in close proximity while avoiding excessive damage (e.g., perforation, stenosis, thrombosis) to carotid arteries, baroreceptors, carotid sinus nerves or other important non-target nerves such as vagus nerve or sympathetic nerves located primarily outside of the carotid septum. CT angiography and ultrasound sonography have been demonstrated to locate carotid bodies in most patients. Thus imaging a carotid body before ablation may be instrumental in (a) selecting candidates if a carotid body is present, large enough and identified and (b) guiding therapy by providing a landmark map for an operator to guide an ablation instrument to the carotid septum, center of the carotid septum, carotid body nerves, the area of a blood vessel proximate to a carotid body, or to an area where carotid body itself or carotid body nerves may be anticipated. Note that although a landmark map may be useful, the need for it may be reduced or eliminated by using devices configured to create and contain an ablation within an intercarotid septum, such as the devices disclosed herein, therefor reducing costly pre-procedural planning and operator dependency on following a landmark map. It may also help exclude patients in whom the carotid body is located substantially outside of the carotid septum in a position close to a vagus nerve, hypoglossal nerve, jugular vein or some other structure that can be endangered by ablation. In one embodiment only patients with carotid body substantially located within the intercarotid septum are selected for ablation therapy. Pre-procedure imaging can also be instrumental in choosing the right catheter depending on a patient's anatomy. For example a catheter with more space between arms can be chosen for a patient with a wider septum.

Once a carotid body is ablated, surgically removed, or denervated, the carotid body function (e.g., carotid body chemoreflex) does not substantially return in humans (in humans aortic chemoreceptors are considered undeveloped). To the contrary, once a carotid sinus baroreflex is removed (such as by resection of a carotid sinus nerve) it is generally compensated, after weeks or months, by the aortic or other arterial baroreceptor baroreflex. Thus, if both the carotid chemoreflex and baroreflex are removed or substantially reduced, for example by interruption of the carotid sinus nerve or intercarotid plexus nerves, baroreflex may eventually be restored while the chemoreflex may not. The consequences of temporary removal or reduction of the baroreflex can be in some cases relatively severe and require hospitalization and management with drugs, but they generally are not life threatening, terminal or permanent. Thus, it is understood that while selective removal of carotid body chemoreflex with baroreflex preservation may be desired, it may not be absolutely necessary in some cases.

### Ablation:

The term “ablation” may refer to the act of altering tissue to suppress or inhibit its biological function or ability to respond to stimulation permanently or for an extended period of time (e.g., greater than 3 weeks, greater than 6 months, greater than a year, for several years, or for the remainder of the patient's life). For example, ablation may involve, but is not limited to, thermal necrosis or irreversible electroporation of target tissue cells.

Carotid Body Ablation (“CBA”) herein refers to ablation of a target tissue wherein the desired effect is to reduce or remove the afferent neural signaling from a chemosensor (e.g., carotid body) or reducing a chemoreflex. Chemoreflex or afferent nerve activity cannot be directly measured in a practical way, thus indexes of chemoreflex such as chemosensitivity can sometimes be used instead. Chemoreflex reduction is generally indicated by a reduction of an increase of ventilation and respiratory effort per unit of blood gas concentration, saturation or blood gas partial pressure change or by a reduction of central sympathetic nerve activity in response to stimulus (such as intermittent hypoxia or infusion of a drug) that can be measured directly. Sympathetic nerve activity can be assessed indirectly by measuring activity of peripheral nerves leading to muscles (MSNA), heart rate (HR), heart rate variability (HRV), production of hormones such as renin, epinephrine and angiotensin, and peripheral vascular resistance. All these parameters are measurable and their change can lead directly to the health improvements. In the case of CHF patients blood pH, blood PCO₂, degree of hyperventilation and metabolic exercise test parameters such as peak VO₂, and VE/VCO₂ slope are also important. It is believed that patients with heightened chemoreflex have low VO₂ and high VE/VCO₂ slope measured during cardiopulmonary stress test (indexes of respiratory efficiency) as a result of, for example, tachypnea and low blood CO₂. These parameters are also related to exercise limitations that further speed up patient's status deterioration towards morbidity and death. It is understood that all these indexes are indirect and imperfect and intended to direct therapy to patients that are most likely to benefit or to acquire an indication of technical success of ablation rather than to proved an exact measurement of effect or guarantee a success. It has been observed that some tachyarrhythmias in cardiac patients are sympathetically mediated. Thus, carotid body ablation may be instrumental in treating reversible atrial fibrillation and ventricular tachycardia.

In the context of this disclosure ablation includes denervation, which means destruction of nerves or their functional destruction, meaning termination of their ability to conduct signals. Selective denervation may involve, for example, interruption of afferent nerves from a carotid body while substantially preserving nerves from a carotid sinus, which conduct baroreceptor signals. Another example of selective denervation may involve interruption of nerve endings terminating in chemo sensitive cells of carotid body, a carotid sinus nerve, or intercarotid plexus which is in communication with both a carotid body and some baroreceptors wherein chemoreflex or afferent nerve stimulation from the carotid body is reduced permanently or for an extended period of time (e.g., years) and baroreflex is substantially restored in a short period of time (e.g., days or weeks). As used herein, the term “ablate” refers to interventions that suppress or inhibit natural chemoreceptor or afferent nerve functioning, which is in contrast to electrically neuromodulating or reversibly deactivating and reactivating chemoreceptor functioning (e.g., with an implantable electrical stimulator/blocker).

Carotid body ablation may include methods and systems for the thermal ablation of tissue via thermal heating mechanisms. Thermal ablation may be achieved due to a direct effect on tissues and structures that are induced by the thermal stress. Additionally or alternatively, the thermal disruption may at least in part be due to alteration of vascular or peri-vascular structures (e.g., arteries, arterioles, capillaries or veins), which perfuse the carotid body and neural fibers surrounding and innervating the carotid body (e.g., nerves that transmit afferent information from carotid body chemoreceptors to the brain). Additionally or alternatively thermal disruption may be due to a healing process, fibrosis, or scarring of tissue following thermal injury, particularly when prevention of regrowth and regeneration of active tissue is desired. As used herein, thermal mechanisms for ablation may include both thermal necrosis or thermal injury or damage (e.g., via sustained heating, convective heating or resistive heating or combination). Thermal heating mechanisms may include raising the temperature of target neural fibers above a desired threshold, for example, above a body temperature of about 37° C. e.g., to achieve thermal injury or damage, or above a temperature of about 45° C. (e.g., above about 60° C.) to achieve thermal necrosis. It is understood that both time of heating, rate of heating and sustained hot or cold temperature are factors in the resulting degree of injury.

In addition to raising temperature during thermal ablation, a length of exposure to thermal stimuli may be specified to affect an extent or degree of efficacy of the thermal ablation. For example, the length of exposure to thermal stimuli may be for example, longer than or equal to about 30 seconds, or even longer than or equal to about 2 minutes. Furthermore, the length of exposure can be less than or equal to about 10 minutes, though this should not be construed as the upper limit of the exposure period. A temperature threshold, or thermal dosage, may be determined as a function of the duration of exposure to thermal stimuli. Additionally or alternatively, the length of exposure may be determined as a function of the desired temperature threshold. These and other parameters may be specified or calculated to achieve and control desired thermal ablation.

In some embodiments, ablation of carotid body or carotid body nerves may be achieved via direct application of ablative energy to target tissue. For example, an ablation element may be applied at least proximate to the target, or an ablation element may be placed in a vicinity of a chemosensor (e.g., carotid body). In other embodiments, thermally-induced ablation may be achieved via indirect generation or application of thermal energy to the target neural fibers, such as through application of an electric field (e.g., radiofrequency, alternating current, and direct current) to the target tissue. For example, thermally induced ablation may be achieved via delivery of a pulsed or continuous thermal electric field to the target tissue such as RF and pulsed RF, the electric field being of sufficient magnitude or duration to thermally induce ablation of the target tissue (e.g., to heat or thermally ablate or cause necrosis of the targeted tissue). Additional and alternative methods and apparatuses may be utilized to achieve ablation, as described hereinafter.

### Endovascular Access:

An endovascular catheter for carotid body ablation via a subclavian artery approach may be delivered into a patient's vasculature via percutaneous introduction into a blood vessel, for example a radial or brachial artery approach to a carotid artery. An endovascular procedure may involve the use of a guide wire, delivery sheath, guide catheter, introducer catheter or introducer. Furthermore, these devices may be steerable and torquable (i.e. able to conduct rotation from proximal to distal end). A carotid access sheath may include lumens for guide wire placement, contrast injection and steerable mechanisms for deflection. Guide wire(s) can be buddy wires placed in the sheath or traverse through the separate limens in sheath or in the catheter itself. Where catheter or sheath lumens are used for contrast injection they also can be used to inject drugs and specifically chemicals that excite or suppress the carotid body. This way carotid body function can be tested during and after a CBM procedure to determine procedure success in stimulating or suppressing carotid body function. Examples of such agents known in medicine and include for example adenosine and dopamine.

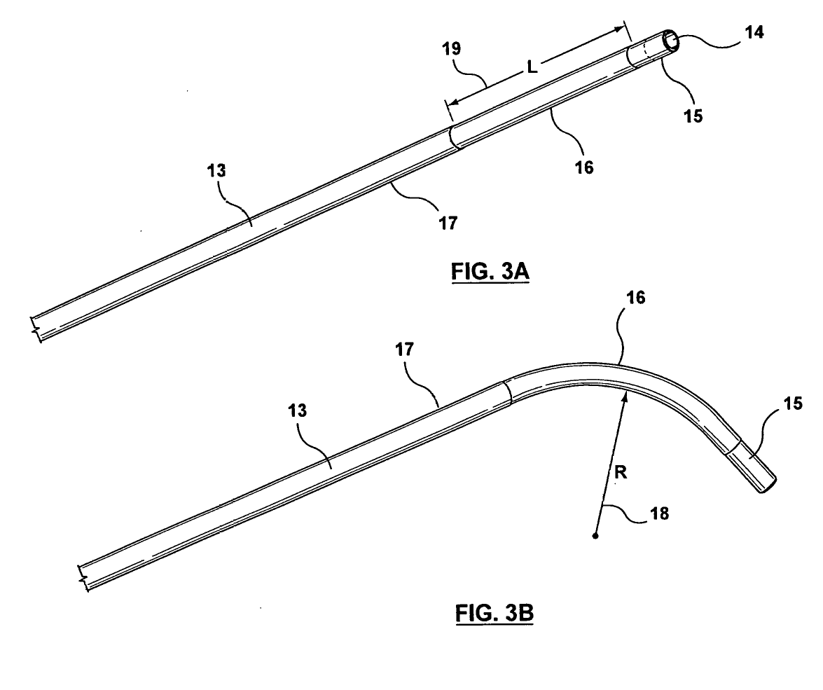

FIG. 3A and FIG. 3B depict a distal end of a carotid access sheath specifically configured for Endovascular Transmural Ablation of a carotid body, which will hereby be referred to as an ETA Carotid Access Sheath 13. The ETA Carotid Access Sheath comprises a central lumen 14 that traverses the length of the sheath from the distal end depicted in FIGS. 3A and 3B to the proximal end (not shown). An ETA Carotid Access Sheath may be sized to accommodate an ablation catheter plus a space sufficient to allow for injection of contrast fluid. The maximum diameter of the sheath is limited by the smallest vessel diameter in which the sheath will be inserted. However, invasiveness of the procedure is minimized as sheath diameter is reduced. For example, the central lumen 14 of the sheath may have a diameter between about 3 French and 12 French (e.g., about 7 French when used with a 6 French ablation catheter). The ETA Carotid Access Sheath 13 may comprise a distal tip 15, a deflectable segment 16 proximal to the distal tip 15, and a non-deflectable segment 17 proximal to the deflectable segment 16. In addition, not shown, is a handle mounted at the proximal end of the catheter with an actuator configured for user-actuated deflection of the deflectable segment 16. A pull wire in communication between the distal tip 15 and the handle mounted actuator at the proximal end is configured to deflect the deflectable segment 16 in response to user actuation. The techniques for constructing a deflectable tipped sheath are known to those skilled in the art, and therefore are not further elaborated. The ETA Carotid Access Sheath is arranged specifically for endovascular transmural ablation of a carotid body in at least one of the following manners: the radius of curvature 18 and length 19 of the deflectable segment are configured for use in the vicinity of the carotid bifurcation with the radius of curvature 18 being between 5 mm and 20 mm, and the length of the deflectable segment 19 being between 10 mm and 25 mm; distal tip 15 may comprise at least one electrode, not shown, configured for at least one of the following: transmural ablation of a carotid body, stimulation of a carotid body, blockade of a carotid body, stimulation of nervous function not associated with a carotid body, and blockade of nervous function not associated with the function of a carotid body, whereby for these specific arrangements the ETA Carotid Access Sheath 13 is used for transmural ablation, and the central lumen 14 is used to place into the region of the carotid bifurcation 31 an additional procedural instrument, the stimulation or blockade is used to locate a preferred position for transmural ablation of a carotid artery, and stimulation or blockade of nervous function not associated with a carotid body is used to avoid damage to important non-target nervous structures such as the vagal nerve.

Alternatively, a guide wire may be delivered through a patient's vasculature to carotid arteries and a sheath may be delivered over the guide wire. The sheath may or may not have steering or deflectable capabilities. For example, if a sheath is delivered over a wire to a common carotid artery and an ablation catheter is delivered through the sheath, deflection may facilitate positioning of the ablation catheter at a target site and reduce unnecessary contact with non-target portions of carotid vasculature, thus reducing risk of dislodging plaque. An ablation catheter may have deflection capabilities to facilitate positioning at a target site, in which case it may not be necessary for a sheath to have deflection capabilities.

### Subclavian Approach

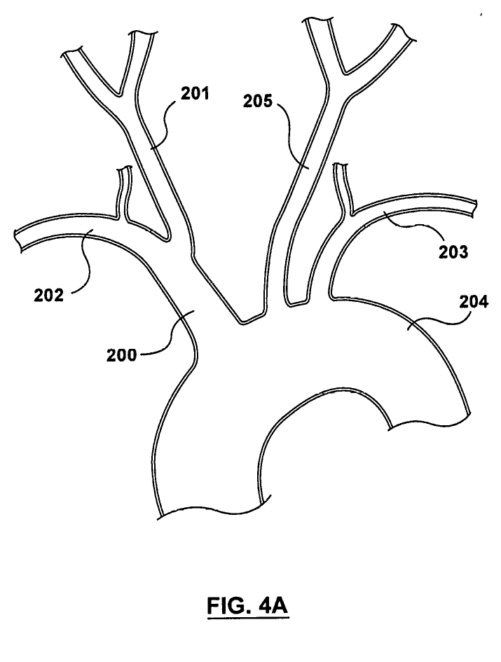

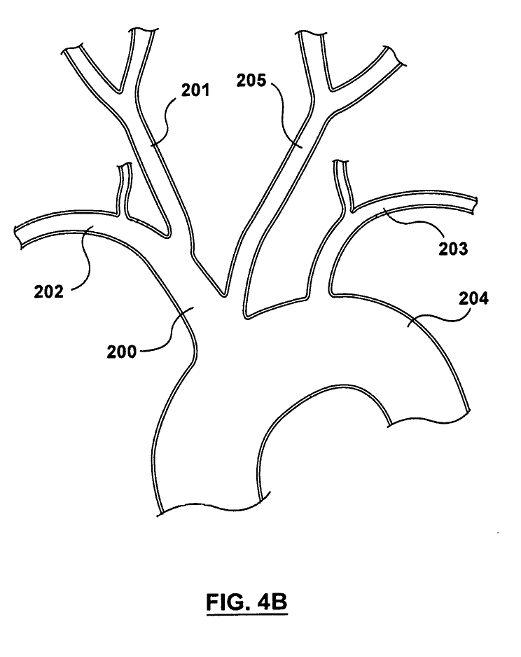

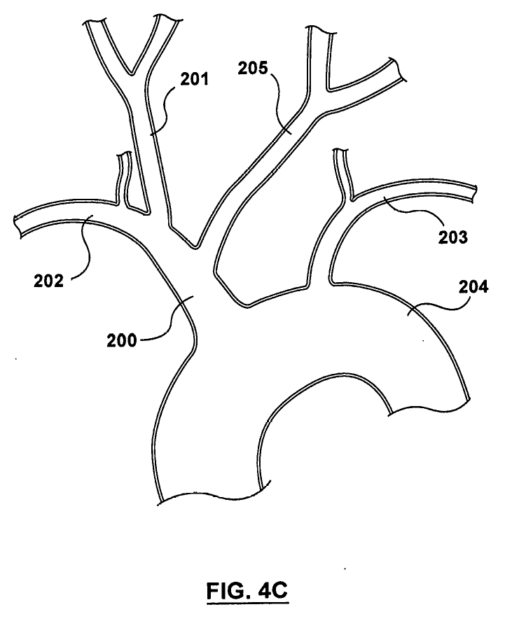

A schematic illustration of the most common aortic arch branching pattern is shown in FIG. 4A. A carotid body ablation catheter may be delivered to a patient's vasculature using radial artery or brachial artery access and advanced to a target region (e.g. in a carotid artery, in a common carotid artery, in an internal carotid artery, in an external carotid artery, at a carotid bifurcation, proximal a carotid septum) by passing the catheter through an axillary and subclavian artery to a innominate artery 200 (a.k.a. brachiocephalic trunk), common carotid artery and to a target region. In most humans a right subclavian artery 202 connects directly to an innominate artery 200 and a right common carotid artery 201 branches from the innominate artery 200. In most humans a left subclavian artery 203 connects directly to an aortic arch 204 and a left common carotid artery 205 branches from the aortic arch 204. Therefore in a typical human patient a right carotid body may be approached by delivering a catheter from a right radial or brachial artery, through a right subclavian artery 202 to an innominate artery 200 and directly to a right common carotid artery 201 and to a target region of a right carotid body. This may be done without entering the aortic arch 204. Access to a region of a left carotid body from a left radial or brachial artery may involve passing the catheter from the left subclavian artery 203 into the aortic arch 204 then up into a left common carotid artery 205. Furthermore, a region of a targeted carotid body on a contralateral side to a radial or brachial artery may be accessed by passing a catheter from a right subclavian artery to an aortic arch then up to the left common carotid artery, or vice versa.

A very common anatomical variant in humans known as a bovine arch describes vasculature in which the left common carotid artery has either a common origin with the innominate artery 200 (see FIG. 4B) or in which the left common carotid artery 205 branches from the innominate artery 200 (see FIG. 4C). In patients having bovine arch structure both a right and a left common carotid artery may be accessed from a right radial or brachial approach without entering the aortic arch.

Transradial catheterization, entry from the wrist, and transbrachial catheterization, entry from the elbow, are known in medical procedures for example to diagnose or treat a disease of an artery or heart. Delivering a carotid body ablation catheter with a subclavian approach may involve either a transradial or transbrachial catheter introduction. These approaches may have a number of benefits compared to transfemoral access procedures. The access site bleeding complications are much less frequent or severe; access site healing is much faster; post procedure ambulation is much faster; less radiopaque contrast agent may be needed; patients may be discharged faster; and costs may be reduced.

Furthermore, an advantage of a transradial or transbrachial catheterization approach over a transfemoral approach for carotid access procedures may include avoiding catheter manipulation at an ostium of an innominate artery from an aortic arch. This area may contain plaque and manipulation of a catheter in this area may increase risk of dislodging plaque and causing embolism. These approaches also may offer an alternative if a transfemoral approach is contraindicated for example due to peripheral vascular disease, infection of the access site or if a patient's aortic arch geometry makes it difficult to access a right common carotid artery. Transradial carotid access for carotid stenting procedures is recently gaining interest.

There are challenges posed by a subclavian approach that may be considered when designing a subclavian carotid body ablation catheter. For example, traversing a catheter over an acute angle from an innominate artery to a right common carotid artery may require specialized techniques, system components or catheter design features. A radial artery diameter is about 2.2 to 3.7 mm, which is narrower than a femoral artery and may impose a design limitation. For example, a device such as a delivery sheath having a diameter of 5 or 6 FR may be ideal for transradial access in a majority of the population, while a 7 or 8FR device may be used in patients having larger radial arteries. Alternatively, since the brachial artery is typically about 4.5 mm a transbrachial access procedure may be performed if the radial artery is too small for the device.

The non-limiting embodiments and concepts disclosed herein may all be useful for a transradial or transbrachial carotid body ablation procedure. For example a carotid body ablation procedure may comprise delivering an endovascular ablation catheter via transradial or transbrachial catheterization wherein the endovascular ablation catheter comprises a structure at a distal region, the structure comprising: two arms configured to couple with a carotid bifurcation; at least one ablation element on one of the arms positioned on the arm such that when the structure is coupled to a carotid bifurcation the at least one ablation element is placed on a target site for carotid body ablation. Furthermore, bipolar RF or energy-directed RF delivered across a carotid septum to ablate tissue in a target region may be suitable ablation modalities for transradial or transbrachial carotid body ablation. It is understood that other ablative energy modalities may also be suitable for transradial or transbrachial carotid body ablation such as for example, ultrasound, microwave, chemical, laser, or cryogenic energy.

A device or system configured for transradial or transbrachial endovascular carotid body ablation may comprise an endovascular ablation catheter and a means to deliver the catheter from a radial or brachial artery to a target region proximate a target tissue (e.g., carotid septum, carotid body, or nerves associated with a carotid body on a right or left side of a patient). The means to deliver the catheter may comprise a means to traverse from a right subclavian artery to a right common carotid artery, which may be close to a right angle or an acute angle, so an energy delivery element may be positioned proximate a target tissue. The means to deliver the catheter may comprise avoiding device manipulation in vasculature proximate an aortic arch where plaque content may be high, which may reduce a risk of embolism. The means to deliver the catheter may comprise a means to traverse from a right subclavian artery to a left common carotid artery in a patient having a bovine arch structure.

Delivering a carotid body ablation catheter from a subclavian artery to a common carotid artery may involve traversing an angle that could be close to a right angle (e.g., between about 80 and 100 degrees) or an acute angle (e.g., less than 90 degrees) and have a radius of curvature less than or equal to about 12 mm (e.g., about 12, 11, 10, 9, 8, 7, 6 mm), and then advancing an interventional device to the target region. Methods and devices known in the art for catheterization across acute angle bifurcations may be used. For example, Suzuki et al. (A Novel Guidewire Approach for handling Acute-Angle Bifurcations: Reversed Guidewire Technique With Adjunctive Use of a Double-Lumen Microcatheter. J Invasive Cardiology 25:1, 2013) describe a technique for advancing guide wire into a vessel over an acute angle. Furthermore, techniques for accessing the common carotid artery for carotid stenting via transradial or transbrachial catheterization exist. Such techniques may be used to deliver a guidewire or guide catheter or delivery sheath to a target region. A carotid body ablation catheter may then be delivered over the guidewire or through the guide catheter or delivery sheath and an energy delivery element may be positioned for carotid body ablation.

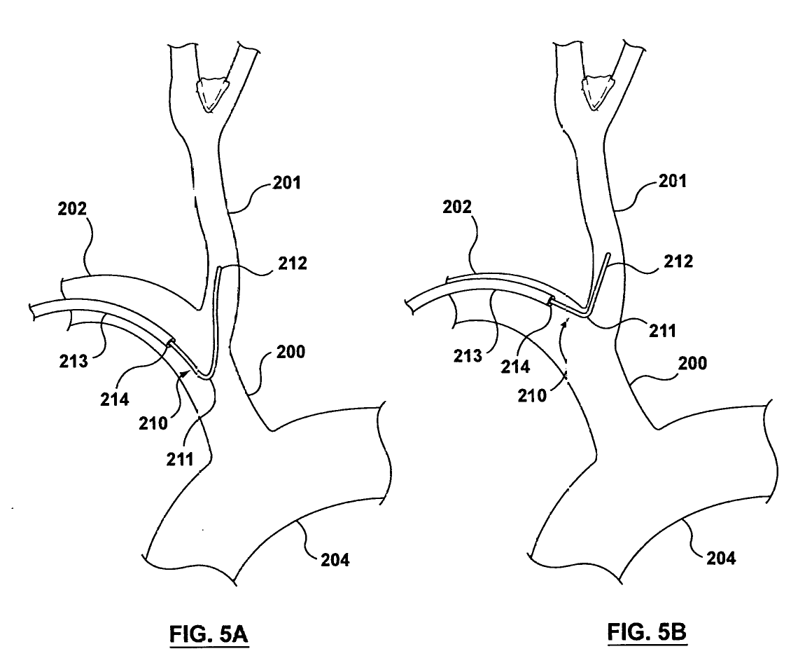

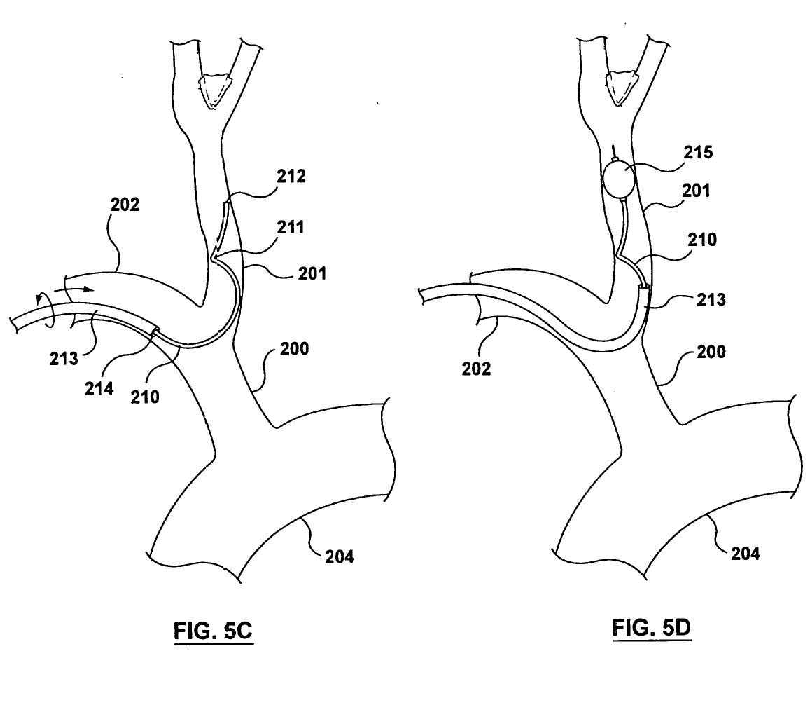

As illustrated in FIGS. 5A to 5D a guidewire 210 having a bend 211 near the distal end may facilitate getting the tip 212 of the guidewire to enter a common carotid artery 201 (or 205 in the case of a patient having a bovine arch anatomy) from an innominate artery 200. The bend may be preformed or actuatable. For example, a guidewire with a bend may have an elastically predisposed, preformed shape that is delivered straight through a sheath 213 and deployed from a distal opening in the sheath 214 when it is positioned near the common carotid artery e.g., 201. Contrast may be injected through the sheath 213 to visualize positioning and the guidewire 210 may be radiopaque. The guidewire may be retracted to engage the preformed bend 211 at the ostium of the common carotid artery 201 from the innominate artery 200 (FIG. 5B). Then, a gentle forward force with rotation of the guidewire shaft may advance the guidewire tip 212 further into the common carotid artery 201 (FIG. 5C). A sheath 213 may then be advanced over the guidewire 210 and into the common carotid artery e.g., 201 (FIG. 5D). Optionally, The guidewire may comprise an inflatable balloon 215 or other anchoring mechanism to hold the guidewire 210 in the common carotid artery 201 while advancing a sheath 213 over it into the common carotid artery so the act of advancing the sheath does not pull the guidewire out of the common carotid artery.

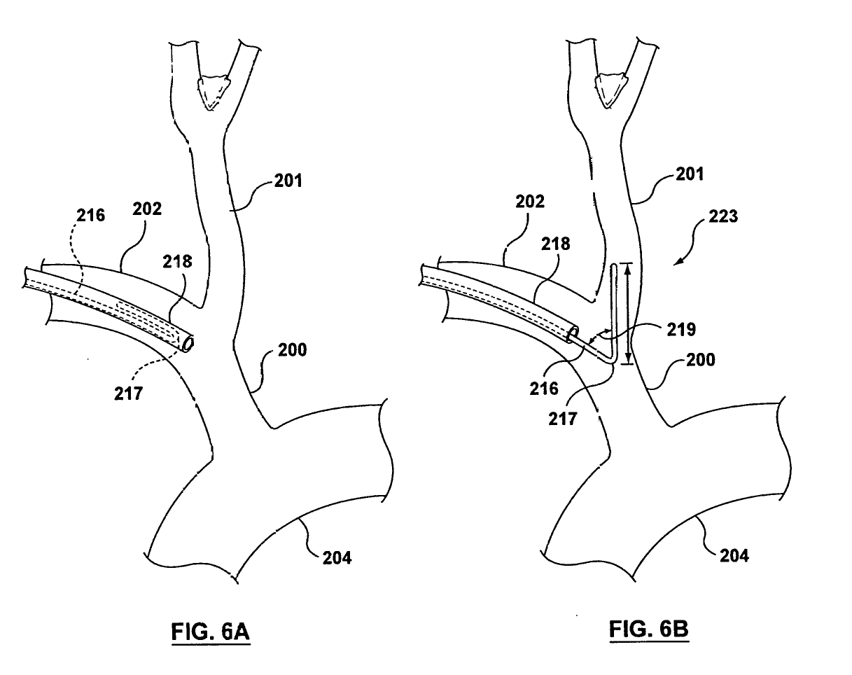

Alternative embodiments of a guidewire as shown in FIGS. 6A and 6B include a guidewire 216 that has distal bent section 217 that is folded while being advanced in a delivery sheath 218 and when positioned near an acute bend the guidewire is advanced out of the sheath and the distal bent section deploys to a predefined bent configuration. As shown in FIG. 6B a predefined configuration may comprise an angle 219 of between about 40° to 80° (e.g., about 45°, 50°, 55°, 60°, 65°), between 45° and 65° and have a distal length 223 of between about 10 to 30 mm. The guidewire may have a wire mandrel core wrapped in a coil and may be made for example from stainless steel or Nitinol.

Another alternative embodiment of a guidewire configured to traverse an acute vascular bend comprises an actuatable bend near the distal end. For example, a bend in a guidewire may be actuated by applying tension to a pull wire that is connected to a distal end wherein a predefined bend is created by applying tension to the pull wire off axis or by controlling deformation with a laser cut tube. Several techniques for manufacturing steerable guidewires are known in the art and may be applied to make a suitable guide wire for this application. Other ways of actuating a bend may include applying heat to a shape memory Nitinol element or applying electrical current to an electroactive polymer.

Another embodiment of a means for traversing an acute vascular bend comprises passing a guidewire or guide catheter through a delivery sheath with a distal end that is configured to direct the guidewire or guide catheter in a direction that is radial to the axis of the delivery sheath. For example, the delivery sheath may have a steerable distal tip, which may bend the distal region having a length of about 20 to 30 mm into a bend having a diameter of curvature of about 12 to 30 mm that deflects the distal tip at an angle of about 45° to 90° from the axis of the shaft. The steerable delivery sheath may be advanced to a position near the common carotid artery where the distal tip is controllably deflected using an actuator and rotated to direct the tip of the sheath into the common carotid artery. A guidewire may then be delivered through the sheath into the common carotid artery. A steerable delivery sheath may be similar to the one shown in FIGS. 3A and 3B.

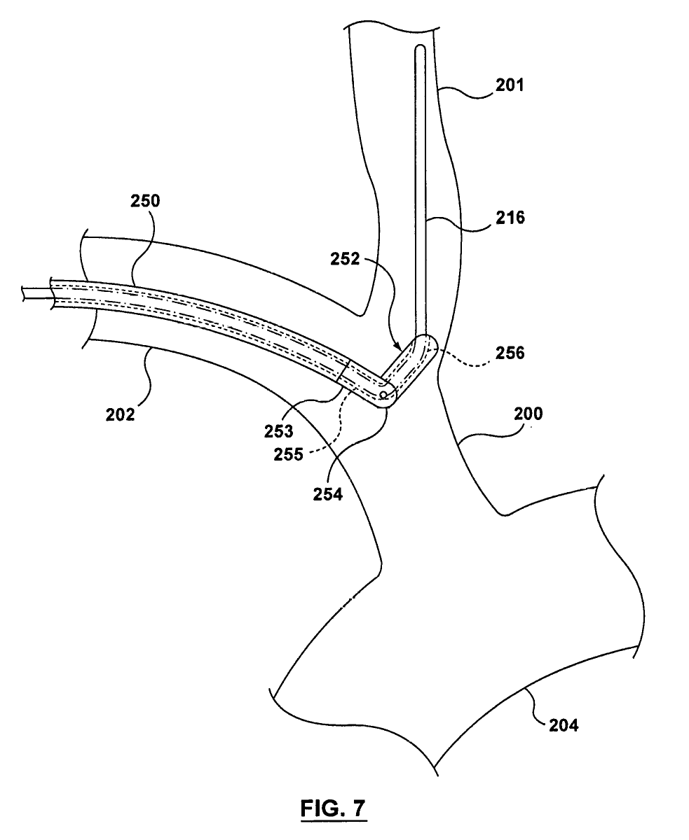

Alternatively, a delivery sheath with a steerable distal end may comprise a pivoting tip as shown in FIG. 7. In this embodiment a delivery sheath 250 comprises a distal end piece 252 that is rigid (e.g. machined from stainless steel) and has a length of about 10 mm. The distal end piece 252 is connected to a connection piece 253 with a pivoting hinge 254. The connection piece 253 is connected to the shaft of the delivery sheath 250. The distal end piece may be pivoted by applying tension to a pull wire (not shown) for example. The connection piece comprises a lumen that is communication with a lumen in the shaft of the sheath. The distal end piece comprises a lumen 255 or channel that may have a curved surface 256. When the distal end piece is pivoted a guidewire 216 may be delivered through the lumens and deflected along the curved surface 256 of the distal end piece as shown in FIG. 7.

Once a guidewire or sheath is positioned in a common carotid artery via transradial or transbrachial catheterization, the sheath or guidewire may then be exchanged for a sheath or guidewire that is more suitable for delivery of an interventional catheter (e.g., carotid body ablation catheter). For example, an ablation catheter delivery sheath may have a larger diameter or may be stiffer; a guidewire for ablation catheter delivery may be stiffer, may be absent the preformed bend or anchor mechanism, or may comprise multiple guidewires.

Furthermore, a means for increasing the radius of curvature at the acute bend may be provided. An increased radius of curvature may facilitate delivery, positioning, torquability, control or use of an ablation catheter that is subsequently delivered through the sheath. Increasing the radius of curvature at the acute bend may comprise distending or reshaping the vasculature in the area of the acute bend. For example, this may involve stretching the vasculature or modifying a vessel's substantially round cross-section to an ovular cross-section.

A means for increasing the radius of curvature at an acute bend may comprise a deployable cage or balloon that reshapes the vasculature.

A means for increasing the radius of curvature at an acute bend may comprise a sheath having a first stiffness that may be transitioned to a second increased stiffness. The sheath may be delivered over a guidewire to cross the acute bend while in a first stiffness state that is relatively flexible and floppy to easily pass over the guidewire. The stiffness of the sheath may then be increased to encourage the sheath to move toward a straighter configuration and increase the radius of curvature particularly at the acute bend. The stiffness of the sheath may be increased for example by increasing hydrostatic pressure in a lumen within the sheath; transitioning a shape-memory alloy such as Nitinol from an easily deformable martensite state to a superelastic austenite state; or inserting a mandrel through a lumen in the sheath to increase radius of curvature of the sheath at the acute bend.

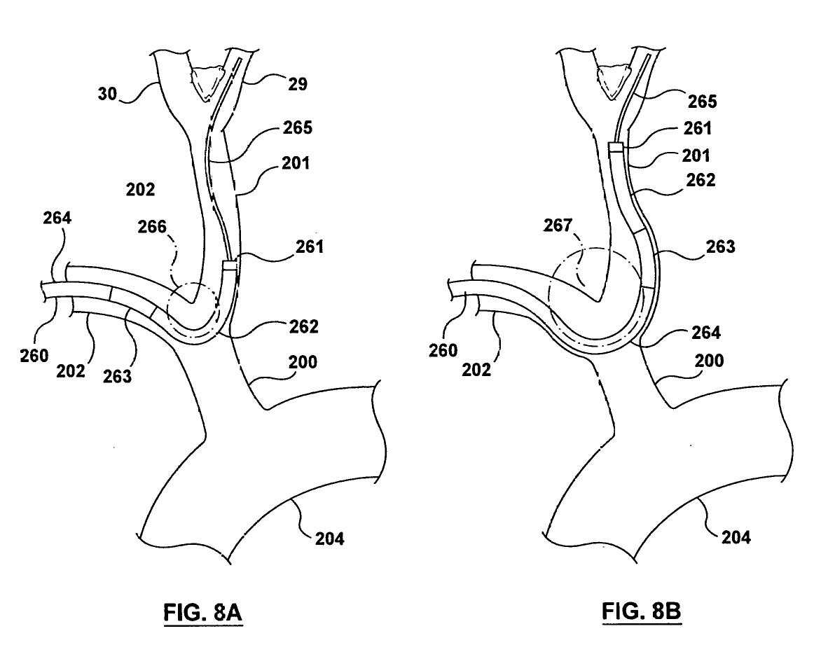

A means for increasing the radius of curvature may involve delivering a second sheath over the guidewire or first sheath that is positioned across an acute vascular pathway (e.g. from a subclavian to common carotid artery), wherein the second sheath has a stiffer durometer than the first sheath. An embodiment of an ablation catheter delivery sheath 260 configured to increase the diameter of curvature of an acute bend, as shown in FIG. 8A and 8B, may comprise an atraumatic tip 261, a relatively flexible region 262, a transition region 263, and a relatively stiff region 264. The atraumatic tip 261 may be a soft polymer with rounded edges that reduces risk of iatrogenic trauma to a vessel as it is advanced, for example over a guidewire 265 or smaller sheath. The relatively flexible region 262 may be made from a flexible polymer and have a length of between about 3 to 5 cm and its function is to bend easily without buckling as it is advanced over a guidewire 265 or smaller sheath traversing an acute bend with a first diameter of curvature 266 between about 12 to 24 mm. The relatively stiffer region 264 may be made from a harder durometer polymer while the transition region 263 may have a durometer between that of the flexible and stiff regions. The transition region 263 may be between about 1 to 2 cm and the stiff region 264 may have a length of at least about 5 cm. As the sheath is advanced the transition region then the relatively stiff region is passed over the acute bend (e.g., between a right subclavian artery 202 and a right common carotid artery 201 or left common carotid artery 205 in patients having bovine arch anatomy) and the increasing stiffness may gently stretch the vasculature or deform its cross-section to increase the diameter of curvature to a second diameter of curvature 267 that is for example between about 20 to 40 mm.

Once a sheath is positioned in a common carotid artery an interventional catheter may be delivered near or to the target region (e.g., near a carotid bifurcation). For example a 5 FR to 7 FR compatible sheath may be delivered from transradial or transbrachial catheterization through a right subclavian artery 202 to a right common carotid artery 201, or a left common carotid artery 205 in patients having a bovine arch structure. A carotid body ablation catheter having features such as but not limited to any of the embodiments or concepts described herein may be used.

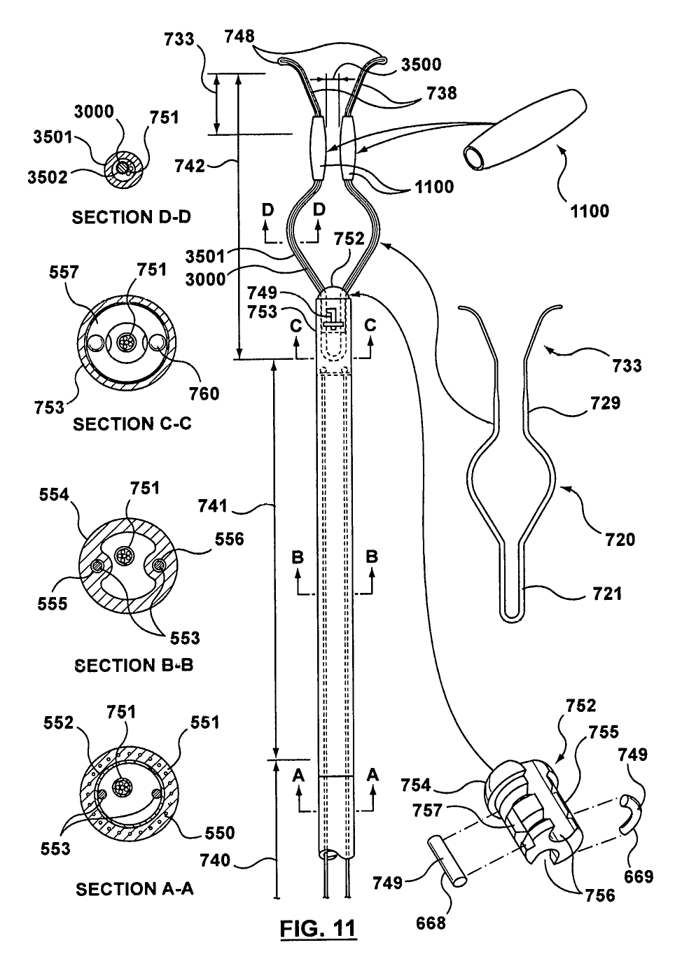

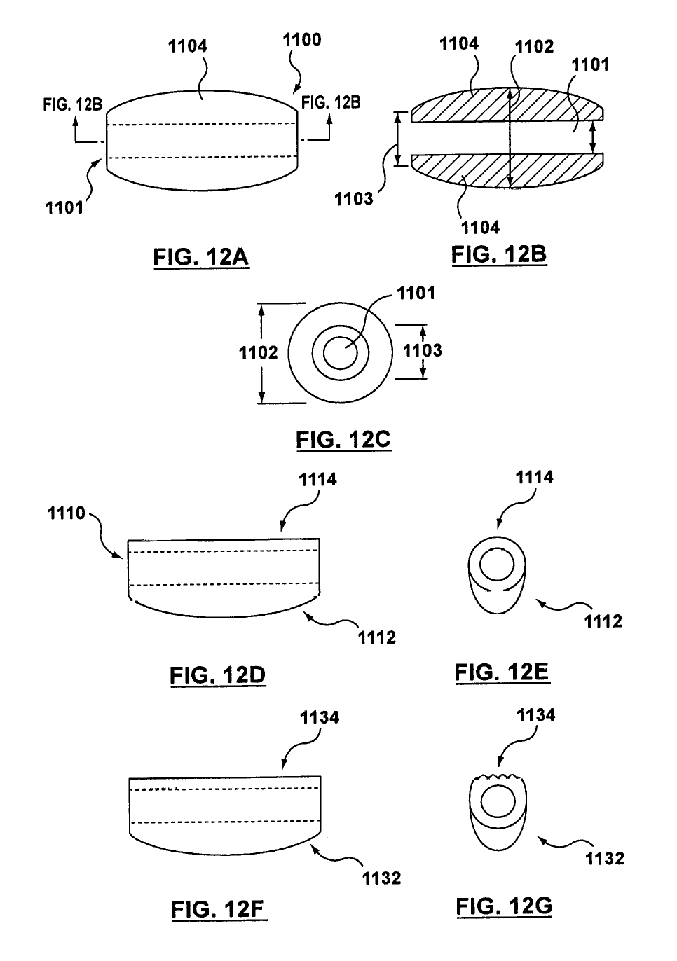

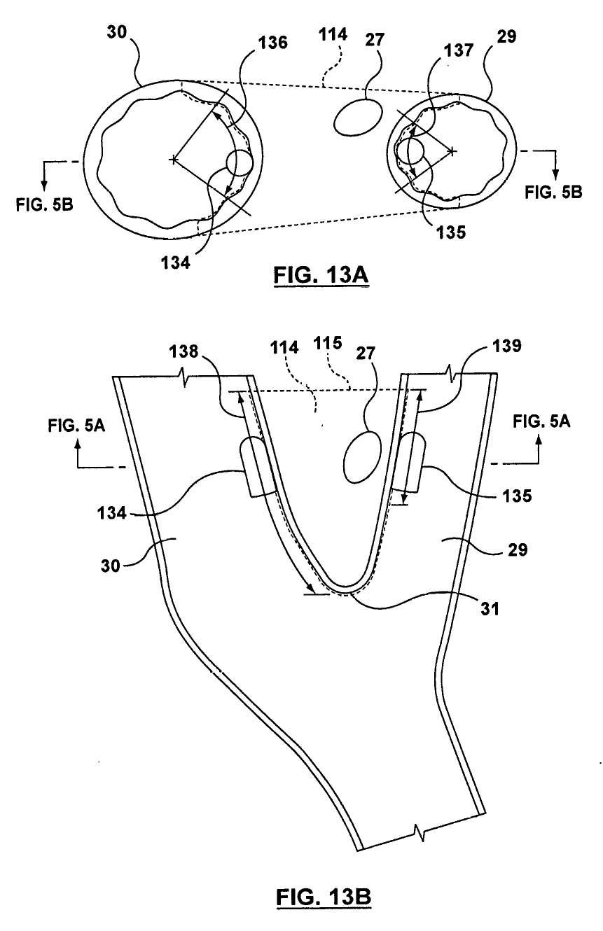

Since a radial artery is smaller than a femoral artery catheter designs that can fit in a 5 FR or 6 FR compatible sheath may be well suited for transradial CB ablation in a majority of patients. For example, a bipolar RF catheter such as the embodiment shown in FIG. 11 may be used. In this design electrode size and shape may contribute to the catheter's ability to fit in a 5FR or 6FR sheath. Reducing electrode size may increase current density in tissue near the electrodes, which could cause unwanted overheating in the localized tissue. To mitigate this risk surface area of electrodes in contact with tissue may be maximized while reducing electrode material that is not in contact with tissue. For example, electrodes such as those shown in FIGS. 12D to 12G maintain a barrel shape and surface area on the side that faces tissue while decreasing the overall diameter of the electrodes.

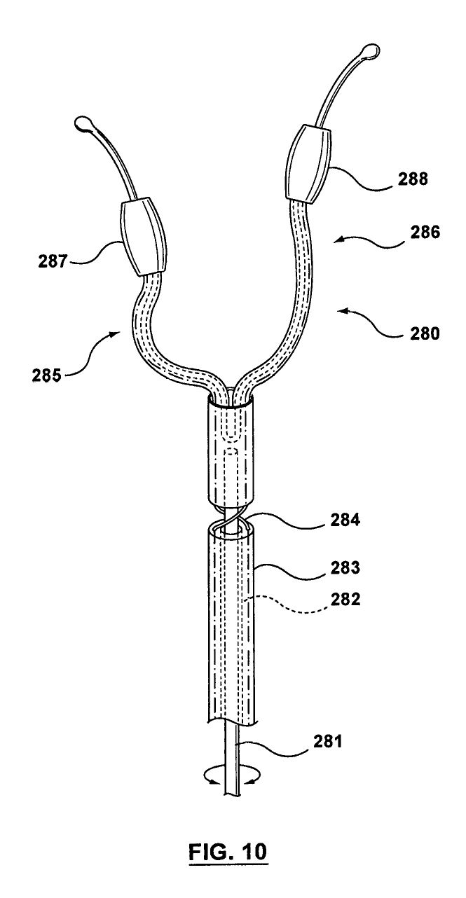

Another embodiment that may allow a reduced size to fit in a 5 FR or 6 FR sheath involves bipolar RF electrodes that are offset such as the embodiment shown in FIG. 10. When this embodiment is retracted into a sheath the electrodes are positioned not side by side but offset along the axial length of the sheath.

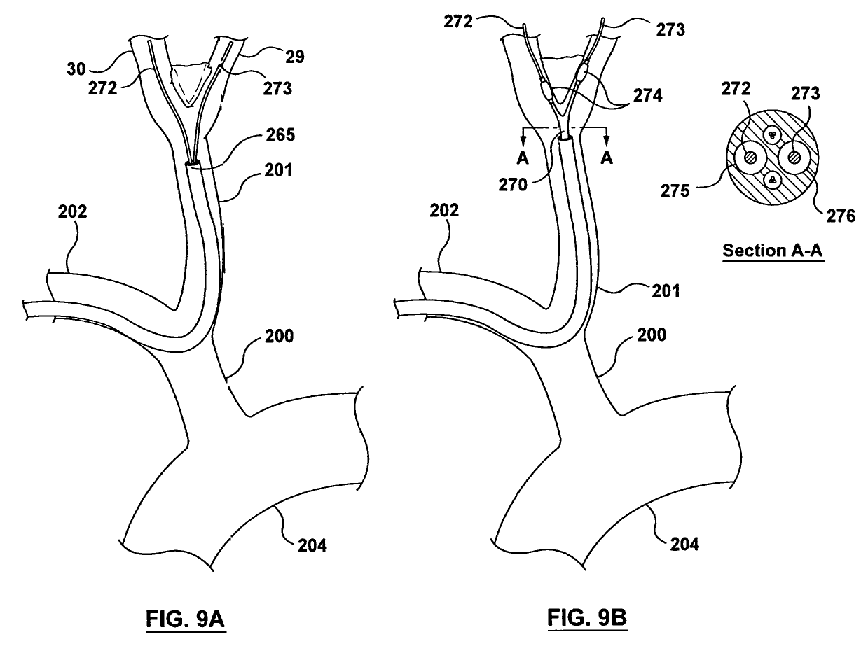

Fine control of catheter rotation may be impeded due to tortuosity of a transradial or transbrachial pathway to a carotid bifurcation. Embodiments that require little or no translation or torque to rotate the distal end may be well suited. For example, as shown in FIGS. 9A and 9B a carotid body ablation catheter 270 may be delivered over two guide wires 272 and 273 delivered from a transradial or transbrachial approach. A first guidewire 273 may be advanced into an external carotid artery 29 and a second guidewire 272 into an internal carotid artery 30. The catheter 270 may comprise two arms each fitted with an electrode 274 (e.g., cylindrical-shaped electrodes, barrel-shaped electrodes such as the electrode shown in FIG. 12A and 12C, or asymmetrical electrodes such as the electrodes shown in FIGS. 12D to 12G) for bipolar RF ablation. The electrodes 274 may be offset along the axial length to optimize electrode diameter and minimize sheath diameter. Each arm may comprise a guidewire lumen 275 and 276 that both continue through the shaft of the catheter to the proximal end. The catheter may be tracked over the two guidewires into position.

An alternative embodiment of a carotid body ablation catheter may comprise a means to facilitate rotation of a distal structure of the catheter particularly in a tortuous vascular approach such as a transradial or transbrachial approach. Rotation of a distal end of a catheter may be facilitated, as shown in FIG. 10, by connecting a distal end structure 280 to a solid wire core 281 that passes through a lumen 282 in a tubular shaft 283. The solid wire core 281 may be rotated within the lumen 282 to rotate the distal end structure 280. Electrical conductors 284 such as RF delivery conductors or sensor conductors may pass through lumens in the tubular shaft (not shown) and have enough slack to allow the distal end structure 280 to rotate relative to the tubular shaft 283 (e.g., up to about) 180°. A handle (not shown) at the proximal end of the catheter may have an actuator that rotates the solid wire core 281 relative to the tubular shaft 283. The solid wire core 281 may have a lubricious coating to reduce friction when rotating. The distal end structure 280 may resemble the distal end structure of many of the embodiments described herein. For example, the distal end structure may comprise two arms 285 and 286 fitted with offset bipolar RF electrodes 287 and 288 as shown and configured for carotid bifurcation coupling. Fine control of rotation may facilitate positioning the arms in the internal and external carotid arteries.

A method of endovascular carotid body ablation may comprise delivering a distal embolic filter in an internal carotid artery via transradial or transbrachial catheterization followed by delivery of an ablation catheter via transfemoral artery catheterization.

### Endovascular Transmural Ablation Precision-Grip Catheters:

An embodiment of a device configured for carotid body ablation via a subclavian artery approach may comprise two arms, herein referred to as Endovascular Transmural Ablation Precision-Grip (ETAP) catheters, which may also be referred to herein as Endovascular Transmural Ablation Forceps (ETAF) catheters. Embodiments of ETAP catheters disclosed herein comprise a distal end and a proximal end, wherein the distal end is inserted into a patient's vasculature and delivered proximate a target site, and the proximal end is maintained outside the patient's body. In some embodiments the distal region of an ETAP catheter comprises ablation element(s) positioned on two arms (which may also be referred to herein as splines, diverging structures, diverging arms, fingers, bifurcated structures, prongs, together as forceps arms, or individually as a forceps arm) in a configuration that positions at least one ablation element in an internal carotid artery and at least one second ablation element in an external carotid artery on an intercarotid septum at a position relative to a target carotid body or nerves associated with a carotid body that is suitable for carotid body ablation. Ablation elements may be, for example, a pair of bipolar radiofrequency electrodes; a pair of bipolar irreversible electroporation electrodes; more than two electrodes; or a single monopolar radiofrequency electrode and second electrode used as current return or to measure properties of target tissue such as electrical impedance, temperature, or blood flow. Apposition of one or both of the ablation elements with an intercarotid septum is achieved by causing a closing force of the arms, for example via resilient forces of the arms or a mechanical actuation means. Structural aspects of catheters may be described herein as bifurcated, but it is not intended that catheter be limited to only two of the structures. For example, when bifurcated is used to describe structural components, at least two are present, and there may be more than two.

FIGS. 13A and 13B illustrate an example of ablation element positioning that may effectively and safely ablate a carotid body 27. 13A shows, outlined with a dashed line, a transverse cross-section of an intercarotid septum 114 bordered by an internal carotid artery 30 and an external carotid artery 29. In this embodiment, a first ablation element 134 is placed in the internal carotid artery 30 in contact with the vessel wall within a vessel wall arc 136 directed toward the external carotid artery; a second ablation element 135 is placed in the external carotid artery 29 in contact with the vessel wall within a vessel wall arc 137 directed toward the internal carotid artery. Each vessel wall arc 136 and 137 is contained within limits of the intercarotid septum 114 and comprises an arc length no greater than about 25% (e.g., about 15 to 25%) of the circumference of the respective vessel. In this example, the ablation elements 134 and 135 may be bipolar radiofrequency electrodes or irreversible electroporation electrodes wherein electrical current is passed from one electrode to the other electrode through the intercarotid septum. Placement of ablation elements as described may facilitate targeted deposition of energy and the creation of an ablation lesion that is contained within the intercarotid septum, thus avoiding injury of non-target nerves that reside outside the septum, and an ablation that is sufficiently large (e.g., with respect to a width dimension, extending approximately from the internal carotid artery to the external carotid artery) to effectively modulate a carotid body or its associated nerves. Specifically, this configuration and placement facilitates deposition of energy along a line between the electrodes and inhibits it in the medial direction (towards the spine).

FIG. 13B shows, outlined with a dashed line, a longitudinal cross-section of an intercarotid septum 114 bordered by an internal carotid artery 30, an external carotid artery 29, a saddle of a carotid bifurcation 31 and a cranial (towards the head) boundary 115 that is between about 10 to 15 mm cranial from the saddle 31. In this example, the first ablation element 134 is placed in the internal carotid artery 30 in contact with the vessel wall within a first range 138; a second ablation element 135 is placed in the external carotid artery 29 in contact with the vessel wall within a second range 139. The first range 138 may extend from the inferior apex of the bifurcation saddle 31 to the cranial boundary 115 of the septum (e.g., about 10 to 15 mm from the bifurcation saddle). The second range 139 may extend from a position about 4 mm superior from the bifurcation saddle 31 to the cranial boundary 115 of the septum (e.g., about 10 or 15 mm from the bifurcation saddle). As an example, an ETAP catheter may be configured to place a distal tip of a 4 mm long electrode in an internal carotid artery about 10 mm from a carotid bifurcation and a distal tip of a second 4 mm long electrode in a corresponding external carotid artery at about 10 mm from the carotid bifurcation. The electrodes 134 and 135 may be equidistant from the saddle 31 or they may be unequal distances from the saddle.

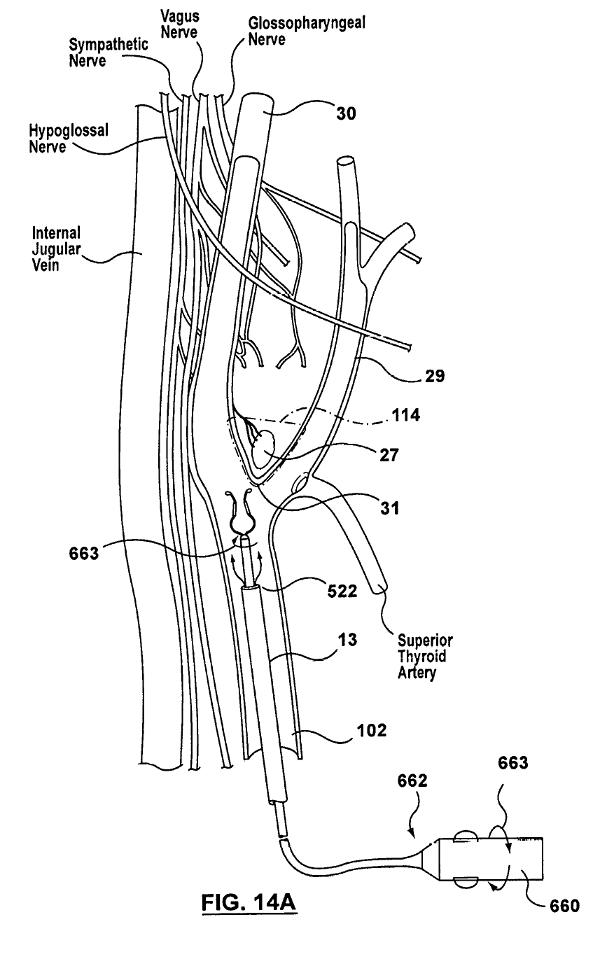

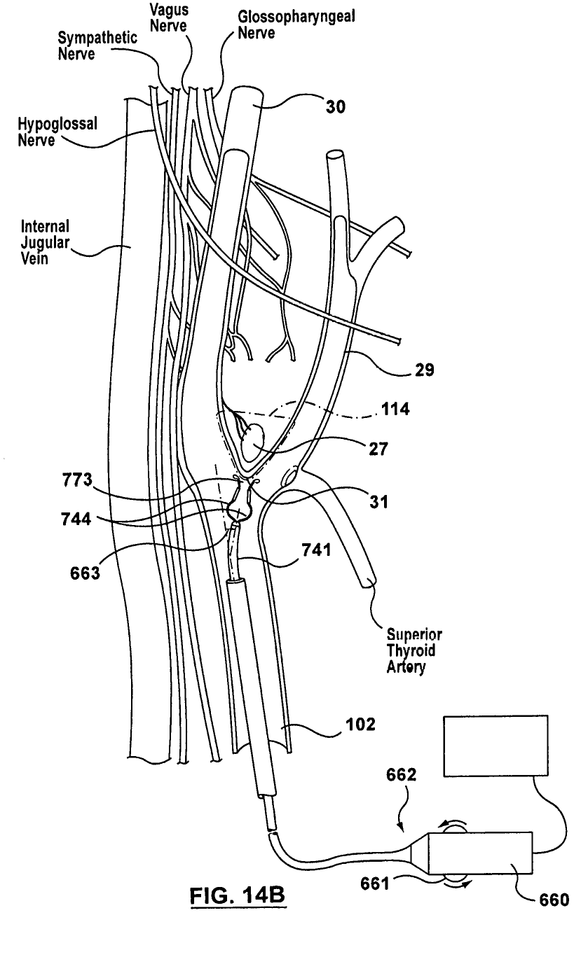

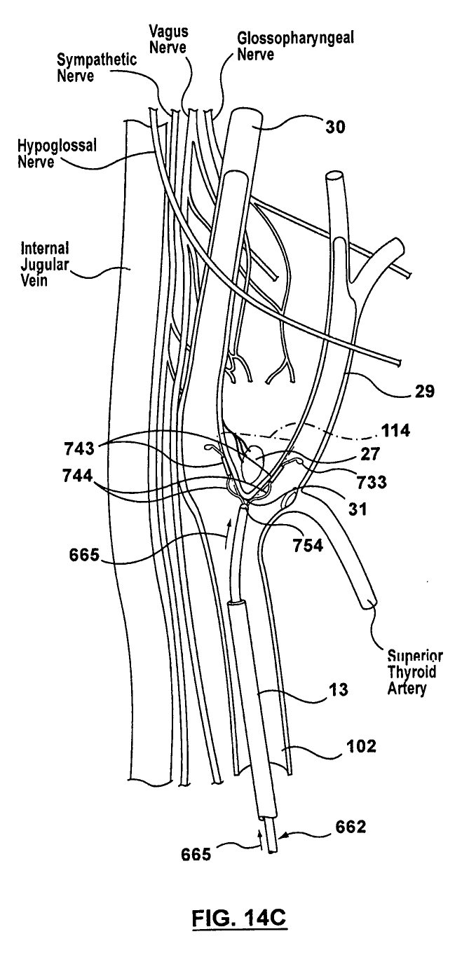

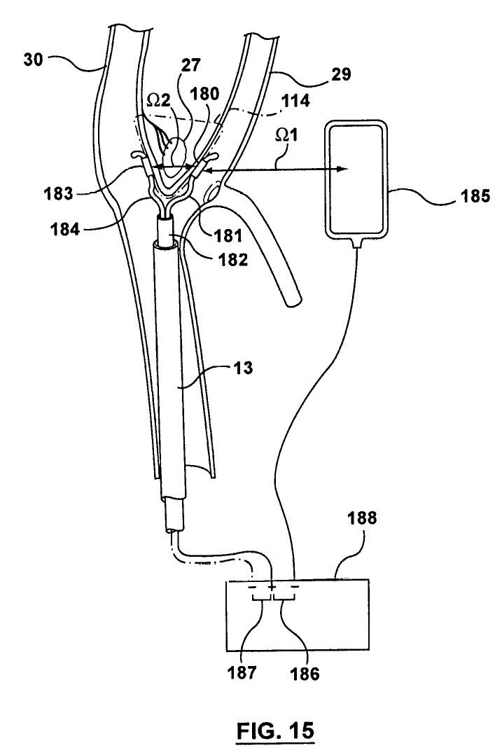

The endovascular carotid septum ablation catheter shown in FIG. 11, shown in use in FIGS. 14A-C, includes first and second diverging arms, the first arm comprising an ablation element and configured so that the ablation element is in contact with a carotid septal wall in an external carotid artery when the catheter is coupled with a common carotid artery bifurcation, the second arm comprising a second ablation element and configured so that the second ablation element is in contact with a carotid septal wall in an internal carotid artery when the catheter is coupled with the bifurcation, as shown in FIG. 14C. The ablation elements are disposed on the arms so that the ablation elements are in contact with the carotid septal walls between the bifurcation and about 4-15 mm cranial to the bifurcation when the catheter is coupled with the bifurcation, as shown in FIG. 14C. In this embodiment each of the ablation elements is disposed on the arms about 4 mm to about 15 mm distal to a distal end of a catheter shaft, the distance being measure along the longitudinal axis of the shaft. This allows the ablation elements to be positioned at desired regions along the septal wall when the catheter is engaging the bifurcation.

In the embodiment shown in FIG. 11, the arms are each configured such that substantially all contact that occurs between the arms and the internal wall surface of the internal and external carotid arteries occurs between the ablation elements and the walls. The arms each have a clearance portion, in this embodiment with a general arch configuration, proximal to the electrode mounting region, as can be seen in FIG. 11, the clearance portion being configured to substantially avoid contact with the walls of the external and internal carotid arteries when the catheter is coupled with a common carotid artery bifurcation such that substantially all contact that occurs between the arms and the walls of the internal and external carotid arteries occurs between the ablation elements and the walls, as shown in FIG. 14C. Each of the clearance portions can be electrically insulated from the ablation element. Each of the clearance portions has an arch configuration. Each of the clearance portions is flexible and resilient such that the clearance portion can be deformed to a straighter configuration for delivery, and is adapted to assume the arch configuration when unconstrained. Each of the clearance portions is configured to make less surface area contact with the wall of the carotid artery than the ablation element, as shown in FIG. 14C. As described herein, the first and second arms are configured to self-align within the internal and external carotid arteries, such as to the positions shown in FIG. 13A. The first and second arms are in substantially the same plane in unstressed configurations, and each arm is flexible so that they are configured to be deflectable out of plane.

FIG. 11 illustrates a distal region of an exemplary endovascular carotid septum ablation catheter that includes first and second diverging arms, the first arm comprising an ablation element and configured so that the ablation element is in contact with a carotid septal wall in an external carotid artery when the catheter is coupled with a common carotid artery bifurcation, the second arm comprising a second ablation element and configured so that the ablation element is in contact with a carotid septal wall in an internal carotid artery when the catheter is coupled with the bifurcation. Once a sheath is positioned via a transradial or transbrachial approach as described earlier, the catheter in FIG. 11 can be positioned for use as is shown in the FIGS. 14A-C.

Mounted on the first and second arms of structural member 3000 in the electrode mounting regions are two electrodes 1100, which may have a barrel shape or curving profile as shown in and described with respect to FIGS. 12A-12C, which facilitates electrode-tissue contact. Alternatively, the electrodes may comprise an asymmetric shape such as shown in FIGS. 12D to 12G. The electrodes 1100 can be approximately 90% gold and 10% platinum, which can be chosen for its electrical, thermal, radiopaque, and machinability properties. The electrodes 1100 are about 4 mm long and have a maximum diameter of about 0.048″, which are able to pass through a 7F sheath next to one another. An electrical conductor (e.g., insulated copper) used to deliver RF energy is electrically connected (e.g., soldered or welded) to each of the first and second electrode 1100 (e.g., in the wall of the electrode or on the inner surface of the lumen 1101 in the electrode). A thermocouple (e.g., T-type) is placed in the lumen 1101 (see FIG. 12B) of each electrode 1100 and its conductors are threaded along the proximal part of the structural member and through the shaft of the catheter. Collectively the RF conductor and thermocouple conductors are 751 in section D-D of FIG. 11. The electrodes 1100 are adhered to the electrode mounting region 3002 on the structural member 3000 with epoxy and insulated from the structural member by a heat shrink insulation such as PET 3502. The structural member, which includes first and second arms, is made from superelastic shape-set Nitinol that has a diameter of about 0.012″ in regions 3001 proximal of the electrodes 1100, which provides sufficient resiliency to apply an electrode apposition force to a carotid septum and self-align when the arms are advanced over a carotid bifurcation to couple with a carotid septum, and yet sufficient flexibility to deform when pulled into a sheath, additional details of which are described in more detail herein. Each of the arms in the structural member is ground down to have a diameter of about 0.006″ in regions 3004 distal to the electrodes 1100, which provides sufficient flexibility for atraumatic contact with vessel walls yet enough resiliency to capture a bifurcation and open the arms as they are passed over a septum, additional details of which are described herein. An electrical insulation 3501 (e.g., thin wall Pebax of about 40D) is applied to each of the arms distal to and proximal to the electrodes 1100 encompassing the electrical conductors 751, the structural member 3000 and the PET insulation and adhered using UV-curable adhesive. The insulation 3501 may be clear to allow UV light to pass through it when curing the adhesive. UV-curable adhesives may also be used to close the distal end of the electrical insulation 3501 and form a dome or ball on the end, which may smoothly glide over a vessel wall with reduced risk of trauma. When the distal structure is assembled as shown there is a space or gap 3500 between the electrodes of about 1 mm +/−0.5 mm measured along a line perpendicular to the longitudinal axis of the catheter shaft, which facilitates advancement of the arms over a septum and allows the arms to deploy smoothly and without getting twisted when advanced from a sheath. The embodiment shown in FIG. 11 may further comprises radiopaque markers 749, a deflectable section near or at the distal end of the shaft controlled by pull wires 553, and a non-deflectable section proximal to the deflectable section. As an example, the elongate catheter shaft may have a braid embedded in its wall to improve transmission of torque and may be approximately 90 to 135 cm long (e.g., about 120 cm) and about 6F diameter, which may be suitable for introduction through a brachial artery. A smaller caliber version such as about 4F or 5F may be suitable for introduction through a radial artery. Modifications of the catheter design to achieve a caliber of about 4F or 5F may include offset electrodes as shown in FIG. 10, or asymmetrically shaped electrodes as shown in FIGS. 12D to 12G. A handle (not shown) may be connected on a proximal end of the elongate shaft.

As shown in use in FIGS. 14A-C, the catheter in FIG. 11 includes ablation elements disposed on the first and second arms so that the ablation elements are in contact with the carotid septal wall in the external and internal arteries between the common carotid bifurcation and about 10-15 mm cranial to the bifurcation when the catheter is coupled with the bifurcation. The ablation elements are in contact with the tissue based on passive contact force. Each of the ablation elements is disposed on the arms about 4 mm to about 15 mm distal to a distal end of a catheter shaft. As in any other embodiment herein, more than two diverging arms may be included in the catheter.

The first and second arms are configured such that substantially all contact that occurs between the arms and the walls of the carotid arteries occurs between the ablation elements and the wall. Substantially all contact includes contact that is at least 60% between the ablation elements and the walls, at least 70% between the ablation elements and the walls, at least 80% between the ablation elements and the walls, at least 90% between the ablation elements and the wall, or more. The first and second arm in the catheter in FIG. 11 include clearance portions proximal to the ablation element, the clearance portions configured to substantially avoid contact with the carotid artery wall when the catheter is coupled with a common carotid artery bifurcation such that substantially all contact that occurs between the arms and the walls of the carotid arteries occurs between the ablation elements and the walls.

In the catheter in FIG. 11, the clearance portions are electrically insulated from the ablation element, and they are shown with arch configuration with a first region that extends away from the catheter shaft axis and a second region that extends back towards the catheter shaft axis. As described in more detail herein, the clearance portions in each arm in the catheter in FIG. 11 is flexible and resilient such that the clearance portion can be deformed to a straighter configuration for delivery, and is adapted to assume the arch configuration when unconstrained. The clearance portions in this embodiment are also configured to make less surface area contact with the walls of the carotid arteries than the ablation element when the catheter is coupled to the bifurcation.

As is described in more detail herein, the first and second arms in the embodiment in FIG. 11 are configured to self-align within the internal and external carotid arteries against the septum. As examples, the first and second arms can comprise a round superelastic wire of between about 0.008″ and about 0.016″ in diameter, such as between about 0.010″ and about 0.014″.

The arms in the embodiment in FIG. 11 are in substantially the same plane in unstressed configurations, and can flexible so that they are configured to be deflectable out of plane, and yet are resilient to allow them to return to the plane. The first and second arms have sufficient resiliency to allow them to move from one stress state to a lower stress state when positioned in contact with the walls of the internal and external carotid arteries. The first and second arms are configured to urge portions of the external carotid arterial wall and the internal carotid artery wall towards each other when positioned in the external and internal carotid arteries and when the catheter is coupled to the bifurcation.

In the embodiment in FIG. 11, the first and second arms have unstressed configurations in which the first and second ablation elements are less than about 6mm apart measured along a line perpendicular to a longitudinal axis of a catheter axis, and can be less than about 4 mm apart measured along a line perpendicular to a longitudinal axis of a catheter axis, and can be less than about 2 mm apart measured along a line perpendicular to a longitudinal axis of a catheter axis.

In FIG. 11 the first and second arms each comprise a distal region distal to the ablation element that extends away from a longitudinal axis of the catheter relative to the ablation element. The distal region is more flexible than a diverging arm region region proximal to the first and second ablation elements. The increased flexibility can be due to a smaller diameter. Additional details of atraumatic tip regions are described herein. The distal regions are each in plane with the respective diverging arm, and are each electrically insulated from the respective ablation element.

The first and second arms of the catheter in FIG. 11 are in substantially the same plane in unstressed configuration, and each of the arms has a free end.

In the embodiment in FIG. 11 the first and second ablation elements are substantially parallel with each other when the first and second arms are in unstressed configurations, but can be angled inward or outward with respect to a longitudinal axis of a catheter shaft. The catheter is also configured for controllable deflection in a first plane that is approximately the plane in which the first and second diverging arms are disposed.

The catheter in FIG. 11 is an example of first and second diverging arms that are symmetrical about a longitudinal axis of the catheter, but they can also be asymmetrical about a longitudinal axis of the catheter. The length of the ablation elements measured along a longitudinal axis of the catheter shaft are the same in this embodiment, but they can be different or have different surfaces areas as described herein. The surface areas of the first and second electrodes are the same but they can be different. The second arm can include a third ablation element different than the second ablation element as is described in more detail herein. The first and second ablation elements are in electrical communication with a generator configured to deliver RF energy to the ablation elements.

The catheter in FIG. 11 includes first and second arms that have substantially the same length, and the lengths in unstressed configurations measured along a longitudinal axis of a catheter shaft are between about 3 mm and about 20 mm, but the arms can have different lengths.

In FIG. 11 a distance between a distal end of the catheter shaft and a distal end of the ablation elements is between about 4 mm and about 15 mm.

In FIG. 11 the ablation elements can have lengths between about 3 and about 10 mm, such as between about 3 mm and about 6 mm, such as about 4 mm.

FIG. 11 shows barrel shaped ablation elements, wherein a central portion of the ablation element is disposed further radially inward than portions of the arm immediately proximal and distal to the ablation element when the arms are in unstressed configurations. The ablation elements also have a greater width dimension along their centers than at the proximal and distal ends. In the embodiment in FIG. 11 the first and second electrodes are disposed at substantially the same distance from a distal end of the catheter shaft measured along a longitudinal axis of the shaft. Each of the ablation elements is also coupled to a temperature sensor configured to sense temperature proximate the ablation element. In alternative embodiments one or both of the arm in the embodiment is configured to be delivered over a guidewire, examples of which are described herein.

The catheter in FIG. 11 is an example of an endovascular carotid septum ablation catheter in comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm comprising a second ablation element and configured so that the second ablation element is in contact with a carotid septal wall in the other of the external carotid artery and an internal carotid artery when the catheter is coupled with a common carotid artery bifurcation, wherein the first and second arms are configured to self-align within the internal and external carotid arteries against the septum.

The catheter in FIG. 11 is an example of an endovascular carotid septum ablation catheter comprising first and second diverging arms with free distal ends, the arms extending generally distally from the catheter; the first arm comprising a first ablation element, the second arm comprising a second ablation element, wherein the first and second arms are, in unstressed configurations, flexible so that they are configured to be deflectable out of plane, and are resilient to allow them to return to the plane.

The catheter in FIG. 11 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, wherein the first arm includes a clearance portion configured to substantially avoid contact with the wall in the one of the external carotid artery and internal carotid artery when the catheter is coupled with a common carotid artery bifurcation such that substantially all contact that occurs between the first arm and the wall of the one of the internal carotid artery or the external carotid artery is made by the ablation element.

The catheter in FIG. 11 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, the first arm comprising a distal region distal to the ablation element that extends away from a longitudinal axis of the catheter relative to the ablation element.

The catheter in FIG. 11 is an example of an endovascular carotid septum ablation catheter comprising first and second diverging arms, the first arm comprising an ablation element and configured so that the ablation element is in contact with a carotid septal wall in one of an external carotid artery and an internal carotid artery when the catheter is coupled with a common carotid artery bifurcation, the second arm configured to be disposed in the other of the internal carotid artery and external carotid artery when the catheter is coupled with the bifurcation, the first arm comprising a distal region distal to the ablation element that is more flexible than a diverging arm region proximal to the ablation element.

The catheter in FIG. 11 is an example of an endovascular carotid septum ablation catheter comprising first and second diverging arms, the first arm comprising no more than a first ablation element and configured so that the ablation element is in contact with a carotid septal wall in one of an external carotid artery and an internal carotid artery when the catheter is coupled with a common carotid artery bifurcation, the second arm comprising no more than a second ablation element and configured to be disposed in the other of the internal carotid artery and external carotid artery when the catheter is coupled with the bifurcation.

The catheter in FIG. 11 is an example of an endovascular carotid septum ablation catheter comprising first and second diverging arms with free distal ends, the arms extending generally distally from the catheter; the first arm comprising a first ablation element, the second arm comprising a second ablation element, and wherein the first and second arms have unstressed configurations in which the first and second ablation elements are less than about 6 mm apart, such as less than about 4 mm apart, and such as less than about 2 mm apart, measured along a line perpendicular to a catheter axis.

The catheter in FIG. 11 is an example of an endovascular carotid septum ablation catheter comprising first and second diverging arms with free distal ends, the arms extending generally distally from the catheter; the first arm comprising a first ablation element, the second arm comprising a second ablation element, wherein the first and second ablation elements are substantially parallel when the arms are in unstressed configurations.

The catheter in FIG. 11 can be modified to be an example of an endovascular carotid septum ablation catheter comprising first and second diverging arms with free distal ends, the arms extending generally distally from the catheter; the first arm comprising a first ablation element, the second arm comprising a second ablation element, at least one of the first and second ablation elements having a distal end angled towards a catheter axis when the first and second arms are unstressed configurations, such as between about 10 and about 30 degrees relative to the catheter axis.

The catheter in FIG. 11 is an example of an endovascular carotid septum ablation catheter comprising first and second diverging arms with free distal ends, the arms extending generally distally from the catheter; the first arm comprising a first ablation element, the second arm comprising a second ablation element, the first and second arms comprising a monolithic structural member.

The catheter in FIG. 11 is an example of an endovascular carotid septum ablation catheter comprising: first and second diverging arms with free distal ends, the arms extending generally distally from the catheter and being in a first plane in unstressed configurations, at least one of the first and second arms comprising an ablation element, wherein the catheter is configured for controllable deflection in the plane.

The catheter in FIG. 11 is an example of an endovascular carotid septum ablation catheter comprising: first and second diverging arms with free distal ends, the arms extending generally distally from the catheter; the first arm comprising a first ablation element, the second arm comprising a second ablation element; and a coating layer, such as an electric insulator, around at least a portion of one of the first and second arms.

The catheter in FIG. 11 is an example of an endovascular carotid body ablation catheter, comprising a structural member comprising a first arm and a second arm, the first arm configured to engage with a wall of the internal carotid artery and the second arm configured to be engaged with a wall of the external carotid artery, a first ablation electrode mounted on the first arm in an electrode-mounting region, and a second ablation electrode mounted on the second arm in a second electrode-mounting region, the first arm, in a region proximal to the electrode-mounting region, has a configuration that extends away from the axis of the structural member and extends toward the axis of the structural member, and the second arm, in a region proximal to the electrode-mounting region, has a configuration that extends away from the axis of the structural member and extends toward the axis of the structural member.

The arm lengths of the catheter in FIG. 11 can be modified such that the catheter is an endovascular carotid septum ablation catheter comprising first and second diverging arms with free distal ends, the arms extending generally distally from the catheters, at least one of the first and second arms comprising an ablation element, wherein a length of the first arm measured along a catheter axis is different than a length of the second arm measured along a catheter axis.

The ablation element(s) on the catheter in FIG. 11 can be modified as described herein such that the catheter is an endovascular carotid septum ablation catheter comprising first and second diverging arms with free distal ends, the arms extending generally distally from the catheters, the first arm comprising at least one energy delivery region, the second arm comprising at least one second energy delivery energy region, wherein that at least one energy delivery region has a tissue contact surface area greater than a tissue contact surface area of the at least one second delivery region.

The arms of the ablation element(s) on the catheter in FIG. 11 can be modified as described herein so that the catheter is an endovascular carotid septum ablation catheter comprising first and second diverging arms with free distal ends, the arms extending generally distally from the catheters, the first arm comprising an ablation element, the first arm comprising a flex circuit including the first ablation element. The second arm can comprise a flex circuit including a second ablation element.

The arms in the catheter in FIG. 11 can be modified as described herein so that the catheter is an endovascular carotid septum ablation catheter comprising first and second diverging arms with free distal ends, the arms extending generally distally from the catheters, at least one of the first and second arms comprising an ablation element, wherein at least one of the first and second arms comprises a guidwire lumen. Both of the arms can also comprise a guidewire lumen.

The catheter in FIG. 11 can be modified as described herein to be an endovascular carotid septum ablation catheter comprising first and second diverging arms with free distal ends, the arms extending generally distally from the catheters, at least one of the first and second arms comprising an ablation element, wherein the first and second arms are secured together distal to a distal end of a catheter shaft.

The catheter in FIG. 11 can be modified as described herein so that it is an endovascular carotid septum ablation catheter comprising first and second diverging arms with free distal ends, the arms extending generally distally from the catheters, at least one of the first and second arms comprising an ablation element, wherein at least one of the arms comprises a pressure or force sensor thereon.

### System

A system has been conceived comprising a catheter, having a means for coupling with an intercarotid septum for carotid body ablation, and an ablation energy console. The system may additionally comprise a connector cable or several cables for connecting the ablation energy console with the catheter, a delivery sheath, or a guide wire. The console may be configured to deliver ablation energy to the catheter. For example, the console may be an electrical signal generator such as a radiofrequency generator or an irreversible electroporation generator. The console may further comprise a user interface that provides the user with a means to select ablation parameters, activate and deactivate an ablation, or to monitor progress of an ablation. The console may further allow a user to select electrical stimulation or blockade used to investigate proximity of an ablation element on the catheter to neural structures. The console may comprise a computer algorithm that controls ablation energy delivery. The algorithm may control energy delivery (e.g., controlled power delivery, ramp time, duration) based on inputs for example, user selected variables, pre-programmed variables, physiologic signals (e.g., impedance, temperature), anatomical features (e.g., intercarotid septum width, presence of plaque, bifurcation angle), or sensor feedback.

Selectable carotid body ablation parameters may include ablation element temperature, duration of ablation element activation, ablation power, ablation element force of contact with a vessel wall, ablation element size, ablation modality, ablation element position within a vessel, or intercarotid septum width.

Pressure or force sensors may be incorporated into any of the catheter embodiments herein, for example they could be mounted to a flex circuit proximate an ablation element, and could be used to verify contact or indicate contact force. Diverging arms with open/close actuation could be actuated to a position that corresponds to a particular contact pressure range. Alternatively, a catheter could be “pushed” against the wall until contact pressure reaches a desired level. Alternatively, a baseline pressure may be chosen when a desirable contact force is visually confirmed, for example vessel distension caused by ablation element contact force may visually appear using an imaging modality such as angiography. A change of pressure or force, within an acceptable range from the baseline, measured by the sensors may indicate appropriate contact force and deviation from this range could indicate an inappropriate contact force. A computer algorithm that controls delivery of ablation energy may discontinue energy delivery if contact force deviates from the appropriate range. Furthermore, a pressure sensor may be used to indicate absolute or relative blood flow and power delivery could be augmented by feedback from the pressure sensor. Alternatively, a temperature sensor, cooled by blood flow, can be used to determine blood flow velocity. Blood flow cooling can be factored into the control algorithms as correction of energy delivery. Also sudden drop of blood flow can indicate spasm of the carotid vessel. Such an abrupt temperature rise will indicate a need to stop or reduce energy delivery instantly. For example, low flow may equal less power and/or power delivery duration, while greater flow may result in more power and/or longer duration. Power of ablation energy delivery may be decreased or duration of energy delivery may be reduced if the flow decreases. Conversely, should the flow increase power or duration may be increased. Alternatively, a pressure sensor may be used to track potential damage to nerves that are to be preserved. Heart rate may be inferred from a pressure sensor through pulsatile flow. The right vagus nerve primarily innervates the sinoatrial node while the left vagus nerve primarily innervates the atrioventricular node. Should either vagus nerve become stimulated, blocked or damaged the patient's heart rate may fluctuate or decline, which may be indicated by the pressure or flow sensor an energy delivery algorithm may stop power delivery or provide a warning accordingly. Similarly, heart function and some gauge of instantaneous heart rate variability may be measured in other ways (e.g., ECG, plethysmography, pulse oximetry) and used by an energy delivery algorithm for safety.

Contact between electrodes and tissue throughout delivery of energy, contact along a full length of an electrode, contact pressure, or stable contact may be important to create a predictable, well controlled ablation. Temperature sensors in each ablation element may be used to indicate characteristics of tissue contact. For example, as energy is applied (e.g., radiofrequency) and tissue is heated, temperature sensors in the ablation elements may be expected to increase as a function of energy delivered and tissue contact. If there is no tissue contact or contact is partial, intermittent, instable or with soft pressure, measured temperature increase may not be as expected (e.g., a lower temperature rise than expected). Temperature measured from multiple sensors may be compared to indicate characteristics of contact. For example if one sensor measures an expected temperature, increase in temperature, or temperature response to energy delivery while a different sensor does not measure an expected result then inconsistent contact may be detected. An algorithm may detect inconsistent ablation element contact and provide a warning and suggest which ablation element requires repositioning.

Tissue impedance, phase or capacitance may be measured between electrodes on each arm of an ETAP catheter in a bipolar arrangement, or between an electrode on one arm and a dispersive electrode on a second arm. Impedance measurement across an intercarotid septum may be used to indicate distance between electrodes, intercarotid septum width, carotid bifurcation angle, position on a bifurcation, tissue characteristics, ablation characteristics, electrode contact with tissue, catheter integrity, presence of plaque (e.g., calcified or atheromatous plaque). An energy delivery algorithm may incorporate impedance feedback, phase changes, or temperature to control delivery of ablation energy. For example, these feedback variables may be used to modulate energy delivery or as a safety cut-off. Ablation energy may be delivered for a predetermined duration of time (e.g., between about 20 and 90s, or in a range of about 20-30s) and energy delivery may be reduced or stopped if there is indication that a traumatic event or a poor ablation is about to happen, such as high temperature or temperature above set point, which may lead to events such as charring or coagulation, or significant movement or poor contact of the electrodes with respect to tissue, which may lead to unpredictable ablation or ablation at a non-target region. Calcified plaque may be detected by high impedance for a given septum width. For example, septum width may be measured using fluoroscopic visualization and if impedance is higher than a predetermined range of normal impedance for the measured septum width then calcified plaque may be present. A computer algorithm may compute presence of plaque based on input septum width and a lookup table of impedance measurements. A bipolar arrangement may be more sensitive to impedance changes and be able to prepare the generator to shut off more quickly than a monopolar arrangement. For example, a bipolar radiofrequency configuration may provide an improved signal to noise ration compared to a monopolar configuration and may provide a clear indication that electrodes are moving. However, an energy delivery control algorithm for either a bipolar or monopolar configuration may incorporate feedback variables for ablation and safety control as discussed herein. For example, prior to charring, which may be indicated by a sharp spike in impedance, several cycles of impedance fluctuation may be measured; if electrode contact with tissue is compromised or electrode position has moved an acute impedance change and simultaneous temperature change at one or both electrodes may be measured; if a catheter is compromised a feedback signal from a temperature sensor may be severed or out of a reasonable range; if a vessel is undergoing spasm impedance and temperature fluctuations as well as power phase changes may be detected simultaneously and in a sinusoidal pattern or may be determined based on hysteresis. Any of these indications may result in a reduction of energy delivery power, power shut off, or a safety warning. Variables such as impedance and temperature may be an indication of a successful ablation. For example, changes in impedance (e.g., value and phase) may be measured when carotid body perfusion is coagulated. This may be an indication that target temperature is exceeding 50-60 C, which may be an indication of technical success. Energy delivery may be stopped or continued for a short amount of time after this occurs to limit a chance that a lesion grows into that hazards medial zone. Another way an energy delivery algorithm may incorporate impedance feedback, phase changes, or temperature to control delivery of ablation energy is to adjust power delivery to meet a set point temperature, impedance, phase or capacitance.

An ETAP or ETAK catheter may be configured for monopolar radiofrequency energy delivery and may comprise only one ablation electrode on an arm and the other arm may not have an electrode but be used for positioning the arms at a carotid bifurcation and in apposition with a target ablation site such as an external carotid artery wall of an intercarotid septum. In this monopolar configuration a dispersive electrode positioned on a patient's skin may compete the radiofrequency circuit. Another embodiment of an ETAP catheter configured for monopolar radiofrequency energy delivery may be constructed the same as embodiments shown in FIG. 11, however an additional dispersive electrode connected to an energy source may be placed on an external surface of a patient and an electrical circuit for ablation may be provided by connecting an energy source to one of the electrodes intended for ablation an the dispersive electrode. As shown in FIG. 15 an active electrode 180 on an arm 181 of an ETAP catheter 182 may be placed, for example, in an external carotid artery 29 in contact with a target ablation site (e.g., vessel wall, intercarotid septum) and a second electrode 183 on a second arm of the ETAP catheter may be placed in the other carotid artery (e.g., internal carotid artery 30), which may facilitate positioning and apposition of the active electrode 180 at a target ablation site. However, the second electrode may be inactive for ablation and, optionally, active for electrical measurements such as tissue impedance, phase, or capacitance. During ablation, a circuit 186 may be made between active electrode 180 and dispersive electrode 185 placed on the patient's skin. The active electrode 180 may deliver radiofrequency current through tissue to dispersive electrode 185. Tissue impedance S21 may be measured during ablation between the active electrode 180 and the dispersive electrode 185 and may be used as a variable to control ablation energy delivery. A circuit 187 between electrodes 180 and 183 may allow a different tissue impedance Ω2 to be measured between these electrodes, which may provide information more specific to the intercarotid septum such as ablation characteristics and electrode contact or motion. Tissue impedance Ω2 may be measured before or after ablation energy is being delivered by transmitting a low power/voltage/current signal between electrodes 180 and 183. Tissue impedance Ω2 may also be measured during ablation, for example, by cycling the ablation energy off periodically (e.g., once every second) for a short duration (e.g., for 1/30 of a second) during which time an impedance measuring signal is delivered between electrode 180 and 183 to obtain tissue impedance Ω2. A control algorithm in an energy console may switch between circuits 186 and 187. Alternatively, two separate radiofrequency energy sources may be used to run circuit 186 and 187. In addition to lower power, voltage, or current for measuring impedance, phase change or capacitance without creating a lesion, circuit 187 may apply a lower frequency, which may capture changes in the underlying tissue (e.g., intercarotid septum) more accurately.

### Methods of Therapy:

A method of using an ETAP catheter with having opening or closing, and deflection actuation may include the following steps:

1. Deliver a sheath (e.g., a 5F to 7F compatible sheath) to a common carotid artery. An over the wire technique or fluoroscopic guidance may be used to deliver a sheath.

2. Deliver the ETAP catheter through the sheath to a common carotid artery. Optionally, the ETAP catheter may be connected to a console to test functionality of the catheter prior to delivering into the patient. For example, electrical current may be delivered through electrical conductors to check if all circuits are functioning properly and sensors, if any, are making reasonable measurements.

3. Deploy a distal working end of the ETAP catheter from the sheath in a closed configuration in the common carotid artery. If the ETAP catheter has a normally-open design the arms may be held in a closed configuration. For example an open/close actuator may be locked in a closed position.

4. Visualize position and rotational plane of the closed arms with respect to a carotid septum. Fluoroscopic techniques may be used to facilitate visualization. For example, contrast solution may be injected through the sheath into the common carotid artery to visualize the vasculature system and radiopaque markers may be placed on the catheter (e.g., on ablation elements and shaft).

5. Rotate/torque the ETAP catheter so arms are approximately in plane with a plane created by the axes of the internal and external carotid arteries.

6. Deflect the distal end of the ETAP catheter with a deflection actuator to aim the distal tip of the catheter at the carotid bifurcation. (note deflection plane is parallel with arms plane) An ETAP catheter configured without controllable deflection may be aimed at a carotid bifurcation using a deflectable sheath.

7. Open the arms with the open/close actuator. An ETAP catheter may be configured to open and close completely, that is, to its full range, upon actuation. Alternatively, an ETAP catheter may be configured to control variable position of the arms from fully open to fully closed. Variable position control may facilitate placement of electrodes, for example, in vasculature have a small bifurcation angle (e.g., less than about 15 degrees).

8. Advance open arms over a septum. The arms may be advanced until the bifurcation of the arms couples with the carotid bifurcation or carina. This may be indicated visually via fluoroscopy, through tactile feedback as a user feels the catheter meeting resistance, or by a contact or force sensor positioned on the distal end of the catheter. Alternatively, arms may be advanced partially, that is, before contact between the bifurcation of the arms and the carotid bifurcation made, for example as indicated visually via fluoroscopy. Partial advancement may be desired if a location of a carotid body or non-target nerves within a septum are known and a desired ablation zone is closer to the carina compared to an ablation zone created when arms are fully advanced. Furthermore, partial advancement may be desired to reduce risk of dislodging plaque that may exist at the carotid bifurcation.

9. Close the arms with the open/close actuator to bring ablation elements (e.g., RF electrodes, electroporation electrodes) into apposition with the septum. Actuation to close the arms may be fully actuated. Elasticity in elastic structural members of the arms may allow closed arms to adjust automatically to various septum thicknesses within a range (e.g., between 2 mm and 15 mm thick or between 4 mm and 10 mm thick) while applying approximately consistent electrode contact force. Alternatively, the degree of closing of the arms may variably controlled, for example, depending on septum thickness or electrode contact force, which may be indicated visually via fluoroscopy or with sensors (e.g., force or impedance sensors). An ETAP catheter may be configured to have arms that are substantially rigid, instead of elastic, so a closing force created by an open/close actuator causes the arms or ablation elements to squeeze an intercarotid septum. This may be advantageous, for example to decrease distance between ablation elements especially when a septum is thick (e.g., greater than 15 mm), which may improve the ability to create an effective ablation.

10. Run an ablation algorithm. For example, an ablation algorithm may be executed by a computerized console and may involve monitoring impedance and temperature, apply ablation energy (e.g., RF or irreversible electroporation) for a predetermined duration and at a predetermined power, shutting off ablation energy if an unwanted scenario occurs such as sudden rise in impedance, sudden large change in temperature, or physiological incidence.

11. Following ablation, open the arms with the open/close actuator to release electrode contact.

12. Retract the arms from the septum into the common carotid artery, for example by pulling the proximal end of the catheter out approximately 2 cm.

13. Close the arms with the open/close actuator. Alternatively, the arms may automatically close when the ETAP catheter is pulled into the sheath.

14. Collect the distal region of the ETAP catheter in the sheath.

15. Remove the sheath and ETAP catheter from the body. Alternatively or optionally, move the sheath and ETAP catheter to the patient's other side to perform a CBM procedure on the contralateral side. This may involve retracting the sheath into the aorta, optionally removing the ETAP catheter from the sheath, introducing a guide wire into the second common carotid artery, and repeating steps for placing the ETAP catheter and ablating.

While the invention has been described in connection with what is presently considered to be the best mode, it is to be understood that the invention is not to be limited to the disclosed embodiment(s). The invention covers various modifications and equivalent arrangements included within the spirit and scope of the appended claims.

What is claimed is:

1. A system configured for endovascular transmural ablation of a carotid body comprising a means for traversing an acute vascular angle configured to pass a guidewire from a right subclavian artery to a common carotid artery and a means to ablate a carotid body.

2. The system of claim 1 further comprising a means for increasing a radius of curvature of the acute angle.

3. The system of claim 2 wherein the means to increase the radius of curvature comprises a sheath having a distal flexible region, a transition region and a relatively stiff region.

4. A method of ablating a carotid body comprising delivering an ablation catheter from a patient's radial or brachial artery through a subclavian artery and to a common carotid artery wherein an ablation element associated with the ablation catheter is positioned proximate a carotid septum.

5. A method of claim 4 comprising delivering a guidewire to the common carotid artery; deploying an anchoring mechanism to anchor a distal end of the guidewire in the common carotid artery; passing a sheath over the guidewire to the common carotid artery; and delivering the ablation catheter through the sheath.

6. A method of claim 4 comprising delivering a guidewire to the common carotid artery through a sheath wherein the sheath comprises an actuatable deflection tip configured to direct the guidewire over an acute bend in a vascular pathway.

7. A method of claim 4 comprising delivering a guidewire to a common carotid artery; delivering a sheath configured to increase a diameter of curvature of an acute bend in a vascular pathway; and delivering the ablation catheter through the sheath.

8. A method of claim 4 comprising delivering a first guidewire to an internal carotid artery and a second guidewire to an external carotid artery; delivering the ablation catheter over the first and second guidewires.

9. A method of claim 8 wherein the ablation element comprises a first and second ablation element positioned un a first and second arm.
